(19) United States
(12) Patent Application Publication
(10) Pub. No.: US 2021/0260036 A1
(43) Pub. Date: Aug. 26, 2021

(54) METHODS OF TREATMENT FOR ALPHA-1 ANTITRYPSIN DEFICIENCY

(71) Applicant: Vertex Pharmaceuticals Incorporated, Boston, MA (US)

(72) Inventors: Carmen BOZIC, Chestnut Hill, MA (US); Brenda CIRINCIONE, Boston, MA (US); Brian J. HARE, Arlington, MA (US); Edward INGENITO, Quincy, MA (US); Sanjeev KUMAR, Lexington, MA (US); Gautham MARIGOWDA, Newton, MA (US); Porntula PANORCHAN, Brookline, MA (US); Mark Christopher PETERSON, Westford, MA (US); David RHEE, Needham, MA (US); David Kent STILES, Danvers, MA (US); Bosheng TIAN, Sudbury, MA (US); Weiyan ZHANG, Quincy, MA (US)

(73) Assignee: Vertex Pharmaceuticals Incorporated

(21) Appl. No.: 17/162,129

(22) Filed: Jan. 29, 2021

Related U.S. Application Data
(60) Provisional application No. 62/967,878, filed on Jan. 30, 2020.
(60) Provisional application No. 63/029,971, filed on May 26, 2020.

(51) Int. Cl. A61K 31/4162; A61K 9/00; A61K 9/20; A61P 43/00

(52) U.S. Cl. CPC A61K 31/4162; A61K 9/0053; A61K 9/2054; A61P 43/00; A61K 9/2009; A61K 9/2027; A61K 9/2031; A61K 9/2013

(57) ABSTRACT

This application describes methods of treating alpha-1 antitrypsin deficiency (AATD) comprising administering Compound I, a deuterated derivative thereof, and/or a pharmaceutically acceptable salt thereof.

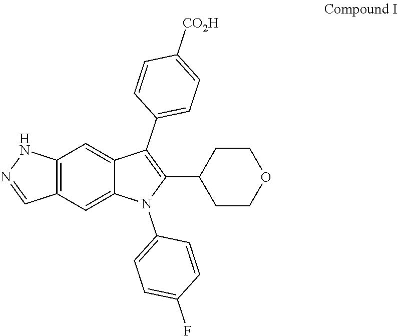 [SMILES: O=C(O)c1ccc(-c2c(C3CCOCC3)n(-c3ccc(F)cc3)c3cc4cn[nH]c4cc23)cc1]

The application also describes pharmaceutical compositions comprising Compound I, a deuterated derivative thereof, and/or a pharmaceutically acceptable salt thereof.

This application claims the benefit of priority to U.S. Provisional Application No. 62/967,878, filed Jan. 30, 2020, and U.S. Provisional Application No. 63/029,971, filed May 26, 2020, the contents of each of which are incorporated by reference herein in their entirety.

Disclosed herein are methods of treating alpha-1 antitrypsin deficiency (AATD) comprising administering Compound I and/or a pharmaceutically acceptable salt thereof.

AATD is a genetic disorder characterized by low circulating levels of alpha-1 antitrypsin (AAT). AAT is produced primarily in the liver and secreted into the blood, although other cell types, including lung epithelial cells, monocytes, macrophages, and neutrophils, produce small amounts of the protein locally (Bergin, et al., Sci Transl Med. 2014; 6(217):217ra1; Geraghty, et al., Am J Respir Crit Care Med. 2014; 190(11):1229-42). AAT inhibits several serine proteinases secreted by polymorphonuclear neutrophils (PMNs; most notably neutrophil elastase, cathepsin G, and proteinase-3) and thus protects organs such as the lung from damage by these proteinases, especially during periods of infection and increased inflammation.

The mutation most commonly associated with AATD involves a substitution of lysine for glutamic acid (E342K) in the SERPINA1 gene that encodes the AAT protein. This mutation, known as the Z mutation, leads to misfolding of the translated protein, which polymerizes within hepatocytes and is not secreted into the bloodstream. Consequently, circulating AAT levels in individuals homozygous for the Z mutation (PiZZ) are markedly reduced; only approximately 15% of mutant Z AAT protein folds correctly and is secreted by hepatocytes into the circulation.

The accumulation of polymerized Z-AAT protein within hepatocytes causes cytotoxicity that can result in neonatal liver disease or progressive liver disease in adulthood that can lead to cirrhosis or liver cancer. The reduced levels of circulating, active AAT result in an imbalance between proteinase and antiproteinase activity, which has its greatest impact in the lung. Consequently, lung tissue is damaged over time resulting in emphysema, which is one pathology occurring in the lungs of subjects with chronic obstructive pulmonary disease (COPD) that contributes to the poorly reversible airflow obstruction that is characteristic of COPD. Emphysema in PiZZ individuals typically manifests in middle age, and usually results in a progressive decline in lung function, a decline in quality of life and shortened lifespan (mean 67 years of age). Piitulainen and Tanash, COPD 2015; 12(1):36-41. PiZZ individuals account for the majority (˜95%) of those with clinically relevant AATD-related lung disease. The accumulation of polymerized Z-AAT protein within hepatocytes causes cytotoxicity that can result in neonatal liver disease or progressive liver disease in adulthood that can lead to cirrhosis or liver cancer.

A milder form of AATD is associated with a mutation in alpha-1 antitrypsin known as the SZ mutation, which results in clinically significant lung disease but not liver disease. Fregonese and Stolk, Orphanet J Rare Dis. 2008; 33:16. As with the ZZ mutation, the deficiency of circulating AAT in subjects with the SZ mutation results in unopposed serine proteinase activity that degrades lung tissue over time and can result in emphysema, particularly in smokers.

The current standard of care for AAT deficient individuals who have or show signs of developing significant lung or liver disease is augmentation therapy (AAT replacement therapy). AAT augmentation therapy involves administration of a pooled, purified human plasma protein concentrate to augment the reduced circulating levels of AAT in subjects with severe AATD. Infusions of the plasma protein have been shown in randomized placebo controlled clinical studies to slow the rate of emphysema progression on CT scans. However, AAT augmentation therapy does not halt lung disease progression and also does not restore the AAT acute phase response which occurs in response to various insults in normal (PiMM) subjects. During the normal AAT acute phase response, plasma AAT levels increase ˜2 fold in response to an insult (such as a pulmonary exacerbation) leading to greater protection of the lung from the increased lung burden of PMN-derived serine proteinases which is associated with increased neutrophilic lung inflammation which occurs during a pulmonary exacerbation. Similarly, although AAT replacement therapy shows promise in slowing the progression of emphysema in subjects with severe AATD, only 2% of the administered drug reaches the lungs. In addition, replacement AAT therapy requires weekly visits for treatment which is burdensome to patients. Thus, there is a continuing need for new and more effective treatments for AATD.

4-[5-(4-fluorophenyl)-6-tetrahydropyran-4-yl-1H-pyrrolo[2,3-f]indazol-7-yl]benzoic acid (Compound I), disclosed in PCT/US2020/032832, filed on May 14, 2020, is being developed as a treatment for AATD. Compound I promotes the proper folding of Z-AAT in multiple cell line models, preventing intracellular Z-AAT protein polymerization and increasing secretion of functionally active AAT. Compound I also facilitates proper folding and secretion of functionally active AAT in transgenic mice engineered to express human Z-AAT. Thus, Compound I has the potential to address both the loss-of-function and the gain-of-function aspects of the Z mutation. By restoring physiological levels of circulating AAT activity, Compound I may reduce the risk of lung disease. By preventing Z-polymer formation in the liver, Compound I may reduce the risk of developing progressive liver disease (fibrosis and cirrhosis).

In some embodiments, the disclosure relates to a compound capable of modulating alpha-1 antitrypsin activity, 4-[5-(4-fluorophenyl)-6-tetrahydropyran-4-yl-1H-pyrrolo[2,3-f]indazol-7-yl]benzoic acid (Compound I), and pharmaceutically acceptable salts thereof. Compound I can be depicted as having the following structure:

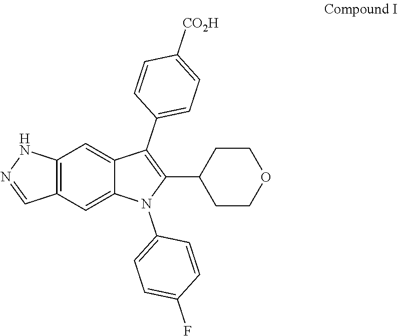 [SMILES: O=C(O)c1ccc(-c2c(C3CCOCC3)n(-c3ccc(F)cc3)c3cc4cn[nH]c4cc23)cc1]

In some embodiments, the disclosure relates to pharmaceutical compositions comprising Compound I and/or at least one pharmaceutically acceptable salt thereof, which compositions may further include at least one additional active pharmaceutical ingredient and/or at least one carrier. In some embodiments, the disclosure provides methods of treating AATD comprising administering Compound I and/or at least one pharmaceutically acceptable salt thereof, optionally as part of a pharmaceutical composition comprising at least one additional component, to a subject in need thereof. In some embodiments, the disclosure provides processes of making Compound I and/or pharmaceutically acceptable salts thereof.

### BRIEF DESCRIPTION OF THE DRAWINGS

FIG. 1 depicts a schematic of a Phase 2 study design for subjects who have never been on augmentation therapy.

FIG. 2 depicts a schematic of a Phase 2 study design for subjects who have been on augmentation therapy at any time.

### DEFINITIONS

“Compound I” as used throughout this disclosure refers to 4-[5-(4-fluorophenyl)-6-tetrahydropyran-4-yl-1H-pyrrolo[2,3-f]indazol-7-yl]benzoic acid, which can be depicted as having the following structure:

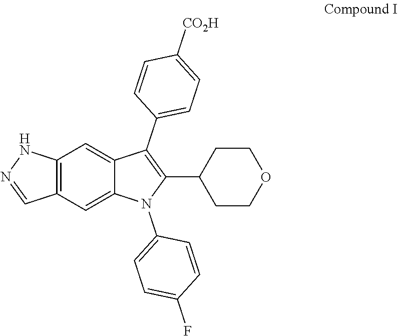 [SMILES: O=C(O)c1ccc(-c2c(C3CCOCC3)n(-c3ccc(F)cc3)c3cc4cn[nH]c4cc23)cc1]

Compound I may be in the form of a pharmaceutically acceptable salt.

As used herein, “AAT” means alpha-1 antitrypsin. As used herein, “AATD” means alpha-1 antitrypsin deficiency.

As used herein, “mutations” can refer to mutations in the SERPINA1 gene (the gene encoding AAT) or the effect of alterations in the gene sequence on the AAT protein. A “SERPINA1 gene mutation” refers to a mutation in the SERPINA1 gene, and an “AAT protein mutation” refers to a mutation that results in an alteration in the amino acid sequence of the AAT protein. A genetic defect or mutation, or a change in the nucleotides in a gene in general, results in a mutation in the AAT protein translated from that gene.

As used herein, a patient who is “homozygous” for a particular gene mutation has the same mutation on each allele.

As used herein, a patient who is “heterozygous” for a particular gene mutation has the particular mutation on one allele, and a different mutation on the other allele.

As used herein, a patient who has the PiZZ genotype is a patient who is homozygous for the Z mutation in the AAT protein.

As used herein, the term “active pharmaceutical ingredient” or “therapeutic agent” (“API”) refers to a biologically active compound.

As used herein, the term “pharmaceutically acceptable salt” refers to a salt form of a compound of this disclosure, wherein the salt is nontoxic. Pharmaceutically acceptable salts of the compounds of this disclosure include those derived from suitable inorganic and organic acids and bases. Pharmaceutically acceptable salts are well known in the art. For example, S. M. Berge, et al. describes pharmaceutically acceptable salts in detail in J. Pharmaceutical Sciences, 1977, 66, 1-19.

As used herein, “ULN” means “upper limit of normal.”

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The terms “patient” and “subject” are used interchangeably and refer to an animal, including a human.

As used herein, the terms “treatment,” “treating,” and the like generally mean the improvement of AATD or its symptoms and/or lessening the severity of AATD or its symptoms in a subject.

As used herein, the term “in combination with,” when referring to two or more compounds, agents, or additional active pharmaceutical ingredients, means the administration of two or more compounds, agents, or active pharmaceutical ingredients to the patient prior to, concurrently with, or subsequent to each other.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. The terms “about” and “approximately” may refer to an acceptable error for a particular value as determined by one of skill in the art, which depends in part on how the values is measured or determined. In some embodiments, the terms “about” and “approximately” mean within (i.e., ±) 20%, 15%, 10%, 5%, 4%, 3%, 2%, 1%, or 0.5% of a given value or range.

One of ordinary skill in the art would recognize that, when an amount of “a compound and/or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form. For example, “100 mg of at least one compound chosen from Compound I and pharmaceutically acceptable salts thereof” includes 100 mg of Compound I and a concentration of a pharmaceutically acceptable salt of Compound I equivalent to 100 mg of Compound I.

As used herein, administration of a “daily” amount of Compound I and/or a pharmaceutically acceptable salt thereof refers to the total amount that is administered in one day but does not limit the frequency of administration per day. The daily amount administered to a patient can be administered once or multiple times in a day, such as twice daily or three times daily (wherein each of multiple administrations comprises administering some amount of Compound I and/or a pharmaceutically acceptable salt thereof that is less than the “daily” amount, given that the “daily” amount refers to the total amount administered in one day). Each administration of Compound I and/or a pharmaceutically acceptable salt thereof can consist of administering Compound I and/or pharmaceutically acceptable salt thereof in the form of a single composition (e.g., a single dosage, such as a single tablet or a single capsule) or in the form of multiple compositions (e.g., multiple dosages, such as multiple (i.e., two or more) tablets and/or capsules).

In some embodiments, the disclosure provides methods of treating AATD with Compound I and/or a pharmaceutically acceptable salt thereof. In some embodiments, Compound I and/or a pharmaceutically acceptable salt thereof is administered daily. In some embodiments, Compound I and/or a pharmaceutically acceptable salt thereof is administered once daily or multiple times daily, such as twice daily or three times daily. In some embodiments, Compound I and/or a pharmaceutically acceptable salt thereof is administered once daily. In some embodiments, Compound I and/or a pharmaceutically acceptable salt thereof is administered twice daily. In some embodiments, Compound I and/or a pharmaceutically acceptable salt thereof is administered three times daily.

In some embodiments, Compound I and/or a pharmaceutically acceptable salt thereof is administered as a single composition. In some embodiments, Compound I and/or a pharmaceutically acceptable salt thereof is administered in multiple compositions (for example, as multiple tablets and/or multiple pills per single administration). Accordingly, in some embodiments, Compound I and/or a pharmaceutically acceptable salt thereof is administered once daily as a single composition. In some embodiments, Compound I and/or a pharmaceutically acceptable salt thereof is administered once daily as multiple compositions, which are administered contemporaneously.

In some embodiments, Compound I and/or a pharmaceutically acceptable salt thereof is administered in a daily amount of 100 mg to 4000 mg. In some embodiments, Compound I and/or a pharmaceutically acceptable salt thereof is administered in a daily amount of 500 mg to 2500 mg. In some embodiments, Compound I and/or a pharmaceutically acceptable salt thereof is administered in a daily amount of 100 mg, 200 mg, 400 mg, 500 mg, 600 mg, 800 mg, 1000 mg, 1200 mg, 1500 mg, 1600 mg, 1800 mg, 2000 mg, 2400 mg, 2500 mg, 2800 mg, 3000 mg, 3200 mg, 3500 mg, 3600 mg, or 4000 mg. In some embodiments, Compound I and/or a pharmaceutically acceptable salt thereof is administered in a daily amount of 100 mg, 200 mg, 400 mg, 500 mg, 600 mg, 800 mg, 1000 mg, 1200 mg, 1500 mg, 1600 mg, 1800 mg, 2000 mg, 2400 mg, 2500 mg, 2800 mg, 3000 mg, 3200 mg, 3500 mg, 3600 mg, or 4000 mg once daily. In some embodiments, Compound I and/or a pharmaceutically acceptable salt thereof is administered twice daily in a daily amount of 100 mg, 200 mg, 400 mg, 500 mg, 600 mg, 800 mg, 1000 mg, 1200 mg, 1500 mg, 1600 mg, 1800 mg, 2000 mg, 2400 mg, 2500 mg, 2800 mg, 3000 mg, 3200 mg, 3500 mg, 3600 mg, or 4000 mg, i.e., Compound I and/or a pharmaceutically acceptable salt thereof is administered in a daily amount (i.e., total amount per day) of 100 mg, 200 mg, 400 mg, 500 mg, 600 mg, 800 mg, 1000 mg, 1200 mg, 1500 mg, 1600 mg, 1800 mg, 2000 mg, 2400 mg, 2500 mg, 2800 mg, 3000 mg, 3200 mg, 3500 mg, 3600 mg, or 4000 mg in two portions (which may be equal or unequal) during a single day. Reference to administration of Compound I and/or a pharmaceutically acceptable salt thereof in an amount of “twice daily” refers to administering an amount of Compound I and/or a pharmaceutically acceptable salt thereof two times in one day, wherein each of the two administrations comprises administration of some amount of Compound I and/or a pharmaceutically acceptable salt thereof that is less than the daily amount, but where the total of these amounts administered in the one day equals the daily amount.

In some embodiments, Compound I and/or a pharmaceutically acceptable salt thereof is administered in a daily amount of 100 mg, 200 mg, 400 mg, 500 mg, 600 mg, 800 mg, 1000 mg, 1200 mg, 1500 mg, 1600 mg, 1800 mg, or 2000 mg twice daily.

In some embodiments, Compound I and/or a pharmaceutically acceptable salt thereof is administered every 8 hours (“q8 h”), every 12 hours (“q12 h”), or every 24 hours (“q24 h”). In some embodiments, Compound I and/or a pharmaceutically acceptable salt thereof is administered every 8 hours (q8 h). In some embodiments, Compound I and/or a pharmaceutically acceptable salt thereof is administered every 12 hours (q12 h). In some embodiments, Compound I and/or a pharmaceutically acceptable salt thereof is administered every 24 hours (q24 h).

In some embodiments, Compound I and/or a pharmaceutically acceptable salt thereof is administered in an amount of 50 mg, 100 mg, 200 mg, 250 mg, 300 mg, 400 mg, 500 mg, 600 mg, 750 mg, 800 mg, 900 mg, 1000 mg, 1200 mg, 1250 mg, 1400 mg, 1500 mg, 1600 mg, 1750 mg, 1800 mg, or 2000 mg every 12 hours (q12 h). In some embodiments, Compound I and/or a pharmaceutically acceptable salt thereof is administered in an amount of 100 mg, 300 mg, or 500 mg every 12 hours (q12 h).

In some embodiments, Compound I and/or a pharmaceutically acceptable salt thereof is administered in an amount of 50 mg, 200 mg, 250 mg, 400 mg, 500 mg, 600 mg, 750 mg, 800 mg, 900 mg, 1000 mg, 1200 mg, 1250 mg, 1400 mg, 1500 mg, 1600 mg, 1750 mg, 1800 mg, or 2000 mg every 24 hours (q24 h). In some embodiments, Compound I and/or a pharmaceutically acceptable salt thereof is administered in an amount of 200 mg, 600 mg, or 1000 mg every 24 hours (q24 h).

In some embodiments, Compound I and/or a pharmaceutically acceptable salt thereof is administered in an amount of 100 mg every 12 hours (q12 h). In some embodiments, Compound I and/or a pharmaceutically acceptable salt thereof is administered in an amount of 300 mg every 12 hours (q12 h). In some embodiments, Compound I and/or a pharmaceutically acceptable salt thereof is administered in an amount of 500 mg every 12 hours (q12 h).

In some embodiments, the disclosure provides pharmaceutical compositions comprising Compound I and/or a pharmaceutically acceptable salt thereof, which compositions may further include at least one additional active pharmaceutical ingredient and/or at least one carrier. In some embodiments, the disclosure provides a pharmaceutical composition comprising at least one compound chosen from Compound I and pharmaceutically acceptable salts thereof, and at least one pharmaceutically acceptable carrier.

Compound I and/or a pharmaceutically acceptable salt thereof can be administered in a single pharmaceutical composition or separate pharmaceutical compositions. Such pharmaceutical compositions can be administered once daily (i.e., every 24 hours (q24 h)) or multiple times daily, such as twice daily. Multiple daily administrations can be administered at any time, such as every 8 hours (q8 h) (i.e., three times per day), or every 12 hours (q12 h) (i.e., twice per day).

In some embodiments, the disclosure provides a pharmaceutical composition comprising 50 mg to 2500 mg of Compound I and/or a pharmaceutically acceptable salt thereof, and at least one pharmaceutically acceptable carrier. In some embodiments, the disclosure provides a pharmaceutical composition comprising 50 mg to 2500 mg of Compound I and/or a pharmaceutically acceptable salt thereof, and at least one pharmaceutically acceptable carrier. In some embodiments, the disclosure provides a pharmaceutical composition comprising 50 mg, 100 mg, 125 mg, 250 mg, 500 mg, 750 mg, 1000 mg, 1250 mg, 1500 mg, 1750 mg, 2000 mg, or 2500 mg of Compound I and/or a pharmaceutically acceptable salt thereof, and optionally at least one pharmaceutically acceptable carrier. In some embodiments, the disclosure provides a pharmaceutical composition comprising 100 mg or 250 mg of Compound I and/or a pharmaceutically acceptable salt thereof, and at least one pharmaceutically acceptable carrier. In some embodiments, the disclosure provides a pharmaceutical composition comprising 100 mg of Compound I and/or a pharmaceutically acceptable salt thereof, and at least one pharmaceutically acceptable carrier. In some embodiments, the disclosure provides a pharmaceutical composition comprising 250 mg of Compound I and/or a pharmaceutically acceptable salt thereof, and at least one pharmaceutically acceptable carrier.

In some embodiments, the patient to whom Compound I and/or a pharmaceutically acceptable salt thereof or a pharmaceutical composition comprising the same is administered is in the fasted state. As used herein, a patient who is in the “fasted state” has abstained from all food and drink (except water) for at least two hours (such as for at least four hours) before and at least two hours after administration of Compound I and/or a pharmaceutically acceptable salt thereof or a pharmaceutical composition comprising the same.

In some embodiments, Compound I, a deuterated derivative thereof, and/or a pharmaceutically acceptable salt thereof is taken with food. In some embodiments, Compound I, a deuterated derivative thereof, and/or a pharmaceutically acceptable salt thereof is taken with fat-containing food.

In some embodiments, the patient to whom Compound I and/or a pharmaceutically acceptable salt thereof or a pharmaceutical composition comprising the same is administered is in the fed state. As used herein, a patient who is in the “fed state” has abstained from all food and drink (except water) for at least eight hours (such as for at least ten hours) before the start of a meal and consumption of the meal is started within 30 minutes of administration of Compound I and/or a pharmaceutically acceptable salt thereof or a pharmaceutical composition comprising the same and the entire meal is consumed in 30 minutes or less. In some embodiments, additional food is not permitted for at least two hours (such as four hours) after administration of Compound I and/or a pharmaceutically acceptable salt thereof or a pharmaceutical composition comprising the same. In some embodiments, water may be consumed without restriction beginning after administration of Compound I and/or a pharmaceutically acceptable salt thereof or a pharmaceutical composition comprising the same. In some embodiments, water may be consumed without restriction beginning at least one hour after administration. In some embodiments, the meal is a high-fat meal, such as a meal containing about 800-1000 calories total and containing about 500-600 calories from fat and/or 55-65 grams of fat. In some embodiments, the meal contains about 800-1000 calories total. In some embodiments, the meal contains about 500-600 calories from fat and/or 55-65 grams of fat. In some embodiments, the meal is not a high-fat meal. In some embodiments, the meal is a low-fat meal, such as a meal containing about 400-500 calories total and containing about 100-125 calories from fat and/or 11-14 grams of fat or a meal containing about 500-600 calories total and containing about 100-125 calories from fat and/or 11-14 grams of fat. In some embodiments, the meal contains about 400-800 calories total. In some embodiments, the meal contains about 400-500 calories total. In some embodiments, the meal contains about 500-600 calories total. In some embodiments, the meal contains about 100-125 calories from fat and/or 11-14 grams of fat. In some embodiments, the meal is a moderate-fat meal, such as a meal containing about 600 calories total and containing about 30-35% fat and/or about 20 g of fat or a meal containing about 500-600 calories total and containing about 30-35% fat and/or about 20 g of fat. In some embodiments, the meal contains about 30-35% fat and/or about 20 g of fat.

In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is started at least 30 minutes (such as 30 minutes, 60 minutes, or 90 minutes) after administration of Compound I and/or a pharmaceutically acceptable salt thereof or a pharmaceutical composition comprising the same and the entire meal is consumed in 30 minutes or less. In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is started at least 60 minutes (such as 60 minutes or 90 minutes) after administration of Compound I and/or a pharmaceutically acceptable salt thereof or a pharmaceutical composition comprising the same and the entire meal is consumed in 30 minutes or less. In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is started at least 90 minutes (such as 90 minutes) after administration of Compound I and/or a pharmaceutically acceptable salt thereof or a pharmaceutical composition comprising the same and the entire meal is consumed in 30 minutes or less. In some embodiments, additional food is not permitted for at least two hours (such as four hours) after administration of Compound I and/or a pharmaceutically acceptable salt thereof or a pharmaceutical composition comprising the same. In some embodiments, water may be consumed without restriction beginning after administration of Compound I and/or a pharmaceutically acceptable salt thereof or a pharmaceutical composition comprising the same. In some embodiments, water may be consumed without restriction beginning at least one hour after administration. In some embodiments, the meal is a high-fat meal, such as a meal containing about 800-1000 calories total and containing about 500-600 calories from fat and/or 55-65 grams of fat. In some embodiments, the meal contains about 800-1000 calories total. In some embodiments, the meal contains about 500-600 calories from fat and/or 55-65 grams of fat. In some embodiments, the meal is not a high-fat meal. In some embodiments, the meal is a low-fat meal, such as a meal containing about 400-500 calories total and containing about 100-125 calories from fat and/or 11-14 grams of fat or a meal containing about 500-600 calories total and containing about 100-125 calories from fat and/or 11-14 grams of fat. In some embodiments, the meal contains about 400-800 calories total. In some embodiments, the meal contains about 400-500 calories total. In some embodiments, the meal contains about 500-600 calories total. In some embodiments, the meal contains about 100-125 calories from fat and/or 11-14 grams of fat. In some embodiments, the meal is a moderate-fat meal, such as a meal containing about 600 calories total and containing about 30-35% fat and/or about 20 g of fat or a meal containing about 500-600 calories total and containing about 30-35% fat and/or about 20 g of fat. In some embodiments, the meal contains about 30-35% fat and/or about 20 g of fat.

In some embodiments, the patient to whom Compound I and/or a pharmaceutically acceptable salt thereof or a pharmaceutical composition comprising the same is administered starts consuming a meal at least 30 minutes (such as 30 minutes, 60 minutes, or 90 minutes) after administration of Compound I and/or a pharmaceutically acceptable salt thereof or a pharmaceutical composition comprising the same and the entire meal is consumed in 30 minutes or less. In some embodiments, the patient to whom Compound I and/or a pharmaceutically acceptable salt thereof or a pharmaceutical composition comprising the same is administered starts consuming a meal at least 60 minutes (such as 60 minutes or 90 minutes) after administration of Compound I and/or a pharmaceutically acceptable salt thereof or a pharmaceutical composition comprising the same and the entire meal is consumed in 30 minutes or less. In some embodiments, the patient to whom Compound I and/or a pharmaceutically acceptable salt thereof or a pharmaceutical composition comprising the same is administered starts consuming a meal at least 90 minutes (such as 90 minutes) after administration of Compound I and/or a pharmaceutically acceptable salt thereof or a pharmaceutical composition comprising the same and the entire meal is consumed in 30 minutes or less. In some embodiments, additional food is not permitted for at least two hours (such as four hours) after administration of Compound I and/or a pharmaceutically acceptable salt thereof or a pharmaceutical composition comprising the same. In some embodiments, water may be consumed without restriction beginning after administration of Compound I and/or a pharmaceutically acceptable salt thereof or a pharmaceutical composition comprising the same. In some embodiments, water may be consumed without restriction beginning at least one hour after administration. In some embodiments, the meal is a high-fat meal, such as a meal containing about 800-1000 calories total and containing about 500-600 calories from fat and/or 55-65 grams of fat. In some embodiments, the meal contains about 800-1000 calories total. In some embodiments, the meal contains about 500-600 calories from fat and/or 55-65 grams of fat. In some embodiments, the meal is not a high-fat meal. In some embodiments, the meal is a low-fat meal, such as a meal containing about 400-500 calories total and containing about 100-125 calories from fat and/or 11-14 grams of fat or a meal containing about 500-600 calories total and containing about 100-125 calories from fat and/or 11-14 grams of fat. In some embodiments, the meal contains about 400-800 calories total. In some embodiments, the meal contains about 400-500 calories total. In some embodiments, the meal contains about 500-600 calories total. In some embodiments, the meal contains about 100-125 calories from fat and/or 11-14 grams of fat. In some embodiments, the meal is a moderate-fat meal, such as a meal containing about 600 calories total and containing about 30-35% fat and/or about 20 g of fat or a meal containing about 500-600 calories total and containing about 30-35% fat and/or about 20 g of fat. In some embodiments, the meal contains about 30-35% fat and/or about 20 g of fat.

In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is completed at least 30 minutes (such as 30 minutes, 60 minutes, or 90 minutes) prior to the administration of Compound I and/or a pharmaceutically acceptable salt thereof or a pharmaceutical composition comprising the same. In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is completed at least 60 minutes (such as 60 minutes or 90 minutes) prior to the administration of Compound I and/or a pharmaceutically acceptable salt thereof or a pharmaceutical composition comprising the same. In some embodiments, the patient to whom Compound I and/or a pharmaceutically acceptable salt thereof or a pharmaceutical composition comprising the same is administered has abstained from all food and drink (except water) for at least eight hours (such as for at least ten hours) before the start of a meal and consumption of the meal is completed at least 90 minutes (such as 90 minutes) prior to the administration of Compound I and/or a pharmaceutically acceptable salt thereof or a pharmaceutical composition comprising the same. In some embodiments, additional food is not permitted for at least two hours (such as four hours) after administration of Compound I and/or a pharmaceutically acceptable salt thereof or a pharmaceutical composition comprising the same. In some embodiments, water may be consumed without restriction beginning after administration of Compound I and/or a pharmaceutically acceptable salt thereof or a pharmaceutical composition comprising the same. In some embodiments, water may be consumed without restriction beginning at least one hour after administration. In some embodiments, the meal is a high-fat meal, such as a meal containing about 800-1000 calories total and containing about 500-600 calories from fat and/or 55-65 grams of fat. In some embodiments, the meal contains about 800-1000 calories total. In some embodiments, the meal contains about 500-600 calories from fat and/or 55-65 grams of fat. In some embodiments, the meal is not a high-fat meal. In some embodiments, the meal is a low-fat meal, such as a meal containing about 400-500 calories total and containing about 100-125 calories from fat and/or 11-14 grams of fat or a meal containing about 500-600 calories total and containing about 100-125 calories from fat and/or 11-14 grams of fat. In some embodiments, the meal contains about 400-800 calories total. In some embodiments, the meal contains about 400-500 calories total. In some embodiments, the meal contains about 500-600 calories total. In some embodiments, the meal contains about 100-125 calories from fat and/or 11-14 grams of fat. In some embodiments, the meal is a moderate-fat meal, such as a meal containing about 600 calories total and containing about 30-35% fat and/or about 20 g of fat or a meal containing about 500-600 calories total and containing about 30-35% fat and/or about 20 g of fat. In some embodiments, the meal contains about 30-35% fat and/or about 20 g of fat.

In some embodiments, the patient to whom Compound I and/or a pharmaceutically acceptable salt thereof or a pharmaceutical composition comprising the same consumes a meal at least 30 minutes (such as 30 minutes, 60 minutes, or 90 minutes) prior to the administration of Compound I and/or a pharmaceutically acceptable salt thereof or a pharmaceutical composition comprising the same. In some embodiments, the patient to whom Compound I and/or a pharmaceutically acceptable salt thereof or a pharmaceutical composition comprising the same consumes a meal at least 60 minutes (such as 60 minutes or 90 minutes) prior to the administration of Compound I and/or a pharmaceutically acceptable salt thereof or a pharmaceutical composition comprising the same. In some embodiments, the patient to whom Compound I and/or a pharmaceutically acceptable salt thereof or a pharmaceutical composition comprising the same is administered consumes a meal at least 90 minutes (such as 90 minutes) prior to the administration of Compound I and/or a pharmaceutically acceptable salt thereof or a pharmaceutical composition comprising the same. In some embodiments, additional food is not permitted for at least two hours (such as four hours) after administration of Compound I and/or a pharmaceutically acceptable salt thereof or a pharmaceutical composition comprising the same. In some embodiments, water may be consumed without restriction beginning after administration of Compound I and/or a pharmaceutically acceptable salt thereof or a pharmaceutical composition comprising the same. In some embodiments, water may be consumed without restriction beginning at least one hour after administration. In some embodiments, the meal is a high-fat meal, such as a meal containing about 800-1000 calories total and containing about 500-600 calories from fat and/or 55-65 grams of fat. In some embodiments, the meal contains about 800-1000 calories total. In some embodiments, the meal contains about 500-600 calories from fat and/or 55-65 grams of fat. In some embodiments, the meal is not a high-fat meal. In some embodiments, the meal is a low-fat meal, such as a meal containing about 400-500 calories total and containing about 100-125 calories from fat and/or 11-14 grams of fat or a meal containing about 500-600 calories total and containing about 100-125 calories from fat and/or 11-14 grams of fat. In some embodiments, the meal contains about 400-800 calories total. In some embodiments, the meal contains about 400-500 calories total. In some embodiments, the meal contains about 500-600 calories total. In some embodiments, the meal contains about 100-125 calories from fat and/or 11-14 grams of fat. In some embodiments, the meal is a moderate-fat meal, such as a meal containing about 600 calories total and containing about 30-35% fat and/or about 20 g of fat or a meal containing about 500-600 calories total and containing about 30-35% fat and/or about 20 g of fat. In some embodiments, the meal contains about 30-35% fat and/or about 20 g of fat.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose, and sucrose), starches (such as corn starch and potato starch), cellulose and its derivatives (such as sodium carboxymethyl cellulose, ethyl cellulose, and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

The pharmaceutical compositions described herein are useful for treating AATD.

Any suitable pharmaceutical compositions known in the art can be used for Compound I and/or pharmaceutically acceptable salts thereof. In some embodiments, the pharmaceutical compositions employed in the therapies of the disclosure are tablets. In some embodiments, the tablets are suitable for oral administration. These compositions and combinations are useful for treating AATD.

In some embodiments, pharmaceutical compositions of the disclosure (including, but not limited to, tablets) comprise Compound I and/or a pharmaceutically acceptable salt thereof and cellulose. In some embodiments, pharmaceutical compositions of the disclosure (including, but not limited to, tablets) comprise Compound I and/or a pharmaceutically acceptable salt thereof and croscarmellose sodium. In some embodiments, pharmaceutical compositions of the disclosure (including, but not limited to, tablets) comprise Compound I and/or a pharmaceutically acceptable salt thereof and sodium stearyl fumarate. In some embodiments, pharmaceutical compositions of the disclosure (including, but not limited to, tablets) comprise Compound I and/or a pharmaceutically acceptable salt thereof and lactose monohydrate. In some embodiments, pharmaceutical compositions of the disclosure (including, but not limited to, tablets) comprise Compound I and/or a pharmaceutically acceptable salt thereof and hypromellose acetate succinate. In some embodiments, pharmaceutical compositions of the disclosure (including, but not limited to, tablets) comprise Compound I and/or a pharmaceutically acceptable salt thereof, cellulose, and croscarmellose sodium. In some embodiments, pharmaceutical compositions of the disclosure (including, but not limited to, tablets) comprise Compound I and/or a pharmaceutically acceptable salt thereof, cellulose, croscarmellose sodium, and lactose monohydrate. In some embodiments, pharmaceutical compositions of the disclosure (including, but not limited to, tablets) comprise Compound I and/or a pharmaceutically acceptable salt thereof, cellulose, croscarmellose sodium, hypromellose acetate succinate, and lactose monohydrate. In some embodiments, pharmaceutical compositions of the disclosure (including, but not limited to, tablets) comprise Compound I and/or a pharmaceutically acceptable salt thereof, cellulose, croscarmellose sodium, lactose monohydrate, hypromellose acetate succinate, and sodium stearyl fumarate.

In some embodiments, a tablet comprising Compound I further comprises a coating. In some embodiments, a tablet comprising Compound I further comprises a coating comprising polyvinyl alcohol (PVA), polyethylene glycol (PEG), titanium dioxide, and talc, which is referred to herein as a “non-functional film coating.” An exemplary embodiment of a tablet comprising 250 mg of Compound I and further comprising a non-functional film coating is shown in Table 2. The non-functional film coating can be applied to the tablet comprising Compound I using traditional tablet film coating processes.

TABLE 2
Exemplary Tablet Comprising 250 mg of Compound I and a
Non-Functional Film Coating.

| | Component | Content (% | Amount per |
|---|---|---|---|
| Component | Function | w/w) | Tablet(mg) |
| Compound I | Active | 38.83 | 250.0 |
| Hypromellose acetate | Carrier | 9.71 | 62.5 |
| succinate | | | |
| Microcrystalline Cellulose | Filler | 25.49 | 164.06 |
| Lactose Monohydrate | Filler | 15.78 | 101.56 |
| Croscarmellose Sodium | Disintegrant | 4.37 | 28.31 |
| Sodium Stearyl Fumarate | Lubricant | 2.91 | 18.75 |
| Non-functional Film Coating | Film Coating | 2.91 | 18.75 |
| Total | | 100 | 643.75 |

In some embodiments, disclosed herein are methods of treating, lessening the severity of, or symptomatically treating AATD in a patient comprising administering an effective amount of a compound, pharmaceutically acceptable salt thereof, or a deuterated analog of any of the foregoing; or a pharmaceutical composition, of this disclosure to a patient, such as a human, wherein said patient has AATD. In some embodiments, said patient has the PiZZ genotype. In some embodiments, said patient has the SZ mutation.

In some embodiments, the disclosure also is directed to methods of treatment using isotope-labelled compound of Compound I, which, in some embodiments, are referred to as Compound I′ or pharmaceutically acceptable salt(s) thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine and chlorine, for example ²H, ³H, ¹³C, ¹⁴C, ¹⁵N, ¹⁸O, ¹⁷O, ³¹P, ³²P, ³⁵S, ¹⁸F and ³⁶Cl, respectively.

The isotope-labelled compounds and salts can be used in a number of beneficial ways. They can be suitable for medicaments and/or various types of assays, such as substrate tissue distribution assays. For example, tritium (³H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (²H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The deuterium (²H)-labelled compounds and salts can manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When discovering and developing therapeutic agents, the person skilled in the art attempts to optimize pharmacokinetic parameters while retaining desirable in vitro properties. It may be reasonable to assume that many compounds with poor pharmacokinetic profiles are susceptible to oxidative metabolism.

Non-limiting embodiments of the disclosure include:

1. A method of treating alpha-1 antitrypsin deficiency comprising administering to a patient in need thereof Compound I:

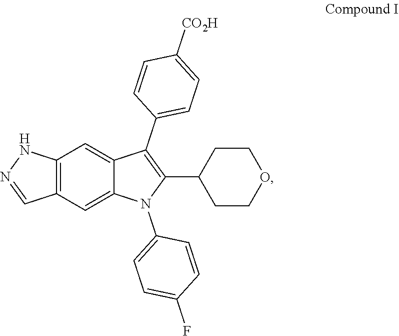 [SMILES: O=C(O)c1ccc(-c2c(C3CCOCC3)n(-c3ccc(F)cc3)c3cc4cn[nH]c4cc23)cc1]

a deuterated derivative thereof, and/or a pharmaceutically acceptable salt thereof in a daily amount of 100 mg to 4000 mg.

2. The method according to embodiment 1, wherein the patient has the PiZZ genotype.

3. The method according to embodiment 1, wherein the patient has an SZ mutation in alpha-1 antitrypsin.

4. The method according to any one of embodiments 1-3, wherein Compound I, a deuterated derivative thereof, and/or a pharmaceutically acceptable salt thereof is administered in a daily amount of 200 mg, 250 mg, 500 mg, 600 mg, 750 mg, 1000 mg, 1250 mg, 1500 mg, 1750 mg, 2000 mg, or 2500 mg.

5. The method according to any one of embodiments 1-4, wherein Compound I, a deuterated derivative thereof, and/or a pharmaceutically acceptable salt thereof is administered in a daily amount of 200 mg, 600 mg, or 1000 mg.

6. The method according to any one of embodiments 1-4, wherein Compound I, a deuterated derivative thereof, and/or a pharmaceutically acceptable salt thereof is administered in a daily amount of 200 mg.

7. The method according to any one of embodiments 1-4, wherein Compound I, a deuterated derivative thereof, and/or a pharmaceutically acceptable salt thereof is administered in a daily amount of 600 mg.

8. The method according to any one of embodiments 1-4, wherein Compound I, a deuterated derivative thereof, and/or a pharmaceutically acceptable salt thereof is administered in a daily amount of 1000 mg.

9. The method according to any one of embodiments 1-8, wherein Compound I, a deuterated derivative thereof, and/or a pharmaceutically acceptable salt thereof is administered multiple times daily.

10. The method according to any one of embodiments 1-9, wherein Compound I, a deuterated derivative thereof, and/or a pharmaceutically acceptable salt thereof is administered every 8 hours (q8 h) or every 12 hours (q12 h).

11. The method according to any one of embodiments 1-8, wherein Compound I, a deuterated derivative thereof, and/or a pharmaceutically acceptable salt thereof is administered once daily.

12. The method according to any one of embodiments 1-3, wherein 100 mg, 250 mg, 300 mg, 500 mg, 750 mg, 1000 mg, 1250 mg, or 1500 mg of Compound I, a deuterated derivative thereof, and/or a pharmaceutically acceptable salt thereof is administered every 12 hours (q12 h).

13. The method according to any one of embodiments 1-3, wherein 100 mg, 300 mg, or 500 mg of Compound I, a deuterated derivative thereof, and/or a pharmaceutically acceptable salt thereof is administered every 12 hours (q12 h).

14. The method according to any one of embodiments 1-13, wherein the method comprises administering Compound I or a deuterated derivative thereof.

15. The method according to any one of embodiments 1-13, wherein the method comprises administering a pharmaceutically acceptable salt of Compound I.

16. The method according to any one of embodiments 1-13, wherein the method comprises administering a pharmaceutical composition comprising Compound I, a deuterated derivative thereof, and/or a pharmaceutically acceptable salt thereof.

17. The method according to embodiment 16, wherein the pharmaceutical composition is a tablet.

18. The method according to embodiment 17, wherein the tablet is suitable for oral administration.

19. The method according to embodiment 18, wherein the tablet for oral administration comprises 100 mg or 250 mg of Compound I, a deuterated derivative thereof, and/or a pharmaceutically acceptable salt thereof.

20. The method according to embodiment 19, wherein the tablet for oral administration comprises 100 mg of Compound I, a deuterated derivative thereof, and/or a pharmaceutically acceptable salt thereof.

21. The method according to embodiment 19, wherein the tablet for oral administration comprises 250 mg of Compound I, a deuterated derivative thereof, and/or a pharmaceutically acceptable salt thereof.

22. The method according to any one of embodiments 16-21, wherein the pharmaceutical composition comprises Compound I, a deuterated derivative thereof, and/or a pharmaceutically acceptable salt thereof, cellulose, croscarmellose sodium, and/or sodium stearyl fumarate.

23. The method according to embodiment 22, wherein the tablet comprises a coating comprising polyvinyl alcohol (PVA), polyethylene glycol (PEG), titanium dioxide, and talc.

24. The method according to any one of embodiments 1-23, wherein the patient is in the fasted state.

25. The method according to any one of embodiments 1-23, wherein the patient is in the fed state.

26. A pharmaceutical composition for use in treating alpha-1 antitrypsin deficiency, wherein the composition comprises Compound I, a deuterated derivative thereof, and/or a pharmaceutically acceptable salt thereof in a daily amount of 100 mg to 4000 mg.

27. The pharmaceutical composition according to embodiment 26, wherein the composition is formulated for administration of Compound I, a deuterated derivative thereof, and/or a pharmaceutically acceptable salt thereof in a daily amount of 200 mg, 250 mg, 500 mg, 600 mg, 750 mg, 1000 mg, 1250 mg, 1500 mg, 1750 mg, 2000 mg, or 2500 mg.

28. The pharmaceutical composition according to embodiment 26, wherein the composition is formulated for administration of Compound I, a deuterated derivative thereof, and/or a pharmaceutically acceptable salt thereof in a daily amount of 200 mg, 600 mg, or 1000 mg.

29. The pharmaceutical composition according to embodiment 26, wherein the composition is formulated for administration of Compound I, a deuterated derivative thereof, and/or a pharmaceutically acceptable salt thereof in a daily amount of 200 mg.

30. The pharmaceutical composition according to embodiment 26, wherein the composition is formulated for administration of Compound I, a deuterated derivative thereof, and/or a pharmaceutically acceptable salt thereof in a daily amount of 600 mg.

31. The pharmaceutical composition according to embodiment 26, wherein the composition is formulated for administration of Compound I, a deuterated derivative thereof, and/or a pharmaceutically acceptable salt thereof in a daily amount of 1000 mg.

32. The pharmaceutical composition according to embodiment 26, wherein the pharmaceutical composition is a tablet.

33. The pharmaceutical composition according to embodiment 32, wherein the tablet is suitable for oral administration.

34. The pharmaceutical composition according to embodiment 33, wherein the tablet for oral administration comprises 100 mg or 250 mg of Compound I, a deuterated derivative thereof, and/or a pharmaceutically acceptable salt thereof.

35. The pharmaceutical composition according to embodiment 34, wherein the tablet for oral administration comprises 100 mg of Compound I, a deuterated derivative thereof, and/or a pharmaceutically acceptable salt thereof.

36. The pharmaceutical composition according to embodiment 34, wherein the tablet for oral administration comprises 250 mg of Compound I, a deuterated derivative thereof, and/or a pharmaceutically acceptable salt thereof.

37. The pharmaceutical composition according to any one of embodiments 26-34, wherein the pharmaceutical composition comprises Compound I, a deuterated derivative thereof, and/or a pharmaceutically acceptable salt thereof, cellulose, croscarmellose sodium, and/or sodium stearyl fumarate.

38. The pharmaceutical composition according to embodiment 34, wherein the tablet comprises a coating comprising polyvinyl alcohol (PVA), polyethylene glycol (PEG), titanium dioxide, and talc.

39. The method according to any one of embodiments 1-23, wherein the patient finishes consuming a meal at least 30 minutes prior to the administration of Compound I, a deuterated derivative thereof, and/or a pharmaceutically acceptable salt thereof.

40. The method according to any one of embodiments 1-23, wherein the patient finishes consuming a meal at least 60 minutes prior to the administration of Compound I, a deuterated derivative thereof, and/or a pharmaceutically acceptable salt thereof.

41. The method according to any one of embodiments 1-23, wherein the patient finishes consuming a meal at least 90 minutes prior to the administration of Compound I, a deuterated derivative thereof, and/or a pharmaceutically acceptable salt thereof.

42. The method according to any one of embodiments 1-23, wherein the patient begins consuming a meal at least 30 minutes after the administration of Compound I, a deuterated derivative thereof, and/or a pharmaceutically acceptable salt thereof.

43. The method according to any one of embodiments 1-23, wherein the patient begins consuming a meal at least 60 minutes after the administration of Compound I, a deuterated derivative thereof, and/or a pharmaceutically acceptable salt thereof.

44. The method according to any one of embodiments 1-23, wherein the patient begins consuming a meal at least 90 minutes after the administration of Compound I, a deuterated derivative thereof, and/or a pharmaceutically acceptable salt thereof.

45. The method according to any one of embodiments 39-44, wherein the patient abstained from all food and drink (except water) for at least eight hours before the start of the meal.

46. The method according to any one of embodiments 39-45, wherein the patient does not consume additional food for at least two hours after the administration of Compound I, a deuterated derivative thereof, and/or a pharmaceutically acceptable salt thereof.

47. The method according to any one of embodiments 39-46, wherein the patient may consume water without restriction beginning after the administration of Compound I, a deuterated derivative thereof, and/or a pharmaceutically acceptable salt thereof.

48. The method according to any one of embodiments 39-47, wherein the meal is a high-fat meal.

49. The method according to any one of embodiments 39-47, wherein the meal is not a high-fat meal.

50. The method according to any one of embodiments 39-47, wherein the meal is a low-fat meal.

51. The method according to any one of embodiments 39-47, wherein the meal is a moderate-fat meal.

52. The method according to any one of embodiments 39-47, wherein the meal contains about 800-1000 calories total.

53. The method according to any one of embodiments 39-47, wherein the meal contains about 500-600 calories from fat and/or 55-65 grams of fat.

54. The method according to any one of embodiments 39-47, wherein the meal contains about 500-800 calories total.

55. The method according to any one of embodiments 39-47, wherein the meal contains about 400-500 calories total.

56. The method according to any one of embodiments 39-47, wherein the meal contains 100-125 calories from fat and/or 11-14 grams of fat 57. The method according to any one of embodiments 39-47, wherein the meal contains about 500-600 calories total.

58. The method according to any one of embodiments 39-47, wherein the meal contains about 30-35% fat and/or about 20 g of fat.

59. The method according to any one of embodiments 1-23, wherein Compound I, a deuterated derivative thereof, and/or a pharmaceutically acceptable salt thereof is taken with food.

60. The method according to any one of embodiments 1-23, wherein Compound I, a deuterated derivative thereof, and/or a pharmaceutically acceptable salt thereof is taken with fat-containing food.

### Example 1: Synthesis of Compound I

### Part A: Synthesis of Starting Materials

### Preparation S1

### 1-(5-(4-fluorophenyl)-7-iodo-6-(tetrahydro-2H-pyran-4-yl)pyrrolo[2,3-f]indazol-1(5H)-yl)-2,2-dimethylpropan-1-one (S1)

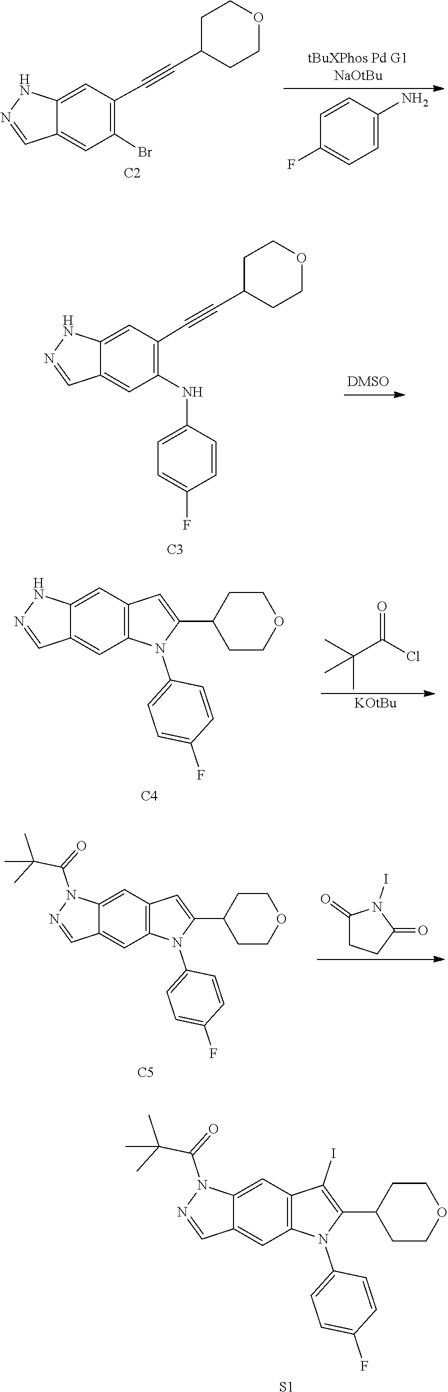

### Steps 1 & 2. Synthesis of 5-(4-fluorophenyl)-6-tetrahydropyran-4-yl-1H-pyrrolo[2,3-f]indazole (C4)

A mixture of 5-bromo-6-(2-tetrahydropyran-4-ylethynyl)-1H-indazole C2 (160 g, 524.3 mmol), 4-fluoroaniline (75 mL, 791.7 mmol), NaOtBu (90 g, 936.5 mmol) in tBuOH (2.1 L) at 40° C. was purged with nitrogen for 10 min. tBuXPhos Pd G1 (10.8 g, 15.7 mmol) was added, and the mixture purged with nitrogen for an additional 10 min. The mixture was heated to 80° C. for 1 h, and then concentrated in vacuo. CH₂Cl₂ (1.5 L), saturated NH₄Cl (1 L), and HCl (62 mL of 6 M, 372.0 mmol) were added. The organic layer was dried with Na₂SO₄, concentrated in vacuo, and re-dissolved in CH₂Cl₂ (160 mL). The mixture was filtered to remove the white inorganic solid. The filtrate was then purified by silica chromatography (Column: 3 kg Silica gel. Gradient: 0-90% EtOAc in heptane) to afford the product contaminated with 4-fluoroaniline. The mixture was dissolved in EtOAc (1.5 L), a washed with 1N HCl (2×250 mL), then brine. The organic layer was dried, and concentrated in vacuo to afford the product as a sticky solid, which was used without further purification (160 g, 91%). LCMS m/z 336.1 [M+H]⁺.

A solution of N-(4-fluorophenyl)-6-(2-tetrahydropyran-4-ylethynyl)-1H-indazol-5-amine C3 in DMSO (550 mL) was heated to 160° C. for 1.5 h. The mixture was cooled, and sat. Na₂CO₃ (500 mL) and water (1.5 L) were added. The mixture was allowed to stir overnight. The resulting grey solid suspension was filtered, and the filter cake was washed with water (×3), then heptane (×3). The filter cake was suspended in TBME (300 mL) and stirred. Solvent was then removed by concentration in vacuo. The resulting solid was dried under vacuum overnight to afford the product (134 g, 76%). ¹H NMR (300 MHz, DMSO-d₆) δ 12.62 (s, 1H), 7.97 (s, 1H), 7.66-7.35 (m, 5H), 7.17 (s, 1H), 6.51 (s, 1H), 3.93-3.75 (m, 2H), 3.24 (td, J=11.3, 5.2 Hz, 2H), 2.82 (dt, J=10.4, 6.3 Hz, 1H), 1.70 (dt, J=10.1, 4.8 Hz, 4H). LCMS m/z 336.1 [M+H]⁺.

### Step 3. Synthesis of 1-[5-(4-fluorophenyl)-6-tetrahydropyran-4-yl-pyrrolo[2,3-f]indazol-1-yl]-2,2-dimethyl-propan-1-one (C5)

To a solution of 5-(4-fluorophenyl)-6-tetrahydropyran-4-yl-1H-pyrrolo[2,3-f]indazole C4 (10 g, 29.8 mmol) in THE (320 mL) at 0° C. was added KOtBu (7.4 g, 65.7 mmol) and the mixture allowed to stir for 5 min. 2,2-dimethylpropanoyl chloride (14.5 mL, 117.9 mmol) was added and the mixture allowed to stir for 1 h. Water (200 mL) and CH₂Cl₂ (250 mL) were added and the mixture extracted with additional dichloromethane (2×50 mL). The organic layer was dried over Na₂SO₄ and concentrated in vacuo. Purification by silica gel chromatography (Gradient: 0-5% EtOAc in Heptane) afforded the product as light yellow solid. 1-[5-(4-fluorophenyl)-6-tetrahydropyran-4-yl-pyrrolo[2,3-f]indazol-1-yl]-2,2-dimethyl-propan-1-one (10.7 g, 83%). ¹H NMR (400 MHz, Chloroform-d) δ 8.69 (s, 1H), 8.07 (s, 1H), 7.39 (dd, J=8.4, 4.9 Hz, 2H), 7.32 (d, J=8.3 Hz, 2H), 7.21 (s, 1H), 6.59 (s, 1H), 4.01 (dd, J=12.0, 4.1 Hz, 2H), 3.37 (t, J=11.7 Hz, 2H), 2.89-2.80 (m, 1H), 1.89 (qd, J=12.2, 4.1 Hz, 2H), 1.78 (d, J=13.0 Hz, 2H), 1.61 (d, J=1.3 Hz, 9H). LCMS m/z 420.3 [M+H]⁺.

### Step 4. Synthesis of 1-[5-(4-fluorophenyl)-7-iodo-6-tetrahydropyran-4-yl-pyrrolo[2,3-f]indazol-1-yl]-2,2-dimethyl-propan-1-one (S1)

1-iodopyrrolidine-2,5-dione (7.4 g, 31.2 mmol) was added portion-wise over 30 min to a solution of 1-[5-(4-fluorophenyl)-6-tetrahydropyran-4-yl-pyrrolo[2,3-f]indazol-1-yl]-2,2-dimethyl-propan-1-one C5 (10.7 g, 25.4 mmol) in CH₂Cl₂ (110 mL). The reaction was stirred at room temperature for 30 min. Purification by silica gel chromatography (Gradient: 0-5% EtOAc in Dichloromethane) resulted in an orange solid, which was triturated with heptane. Water (250 mL) was then added, and the mixture stirred vigorously for 30 min. The solid was filtered, washed with excess water then dissolved in CH₂Cl₂ (250 mL). The solution was washed with water (250 mL) and the organic phase dried (phase separator) and concentrated in vacuo to afford the product as a light tan solid (11.7 g, 84%). ¹H NMR (400 MHz, Chloroform-d) δ 8.63 (s, 1H), 8.08 (s, 1H), 7.37-7.30 (m, 4H), 7.08 (s, 1H), 4.04 (dd, J=11.7, 4.2 Hz, 2H), 3.38 (t, J=11.8 Hz, 2H), 3.07 (t, J=12.6 Hz, 1H), 2.43 (qd, J=12.5, 4.3 Hz, 2H), 1.62 (s, 9H). LCMS m/z 546.33 [M+H]⁺.

### Alternative Preparation of 1-[5-(4-fluorophenyl)-7-iodo-6-tetrahydropyran-4-yl-pyrrolo[2,3-f]indazol-1-yl]-2,2-dimethyl-propan-1-one (S1)

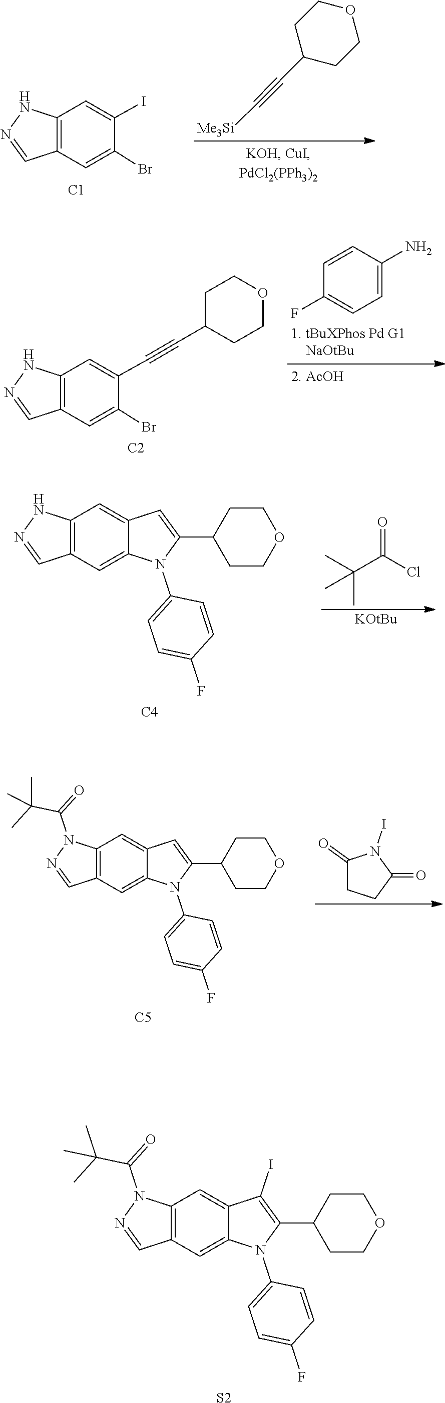

### Step 1. Synthesis of 5-bromo-6-(2-tetrahydropyran-4-ylethynyl)-1H-indazole (C2)

To reactor A under N₂ was charged 5-bromo-6-(2-tetrahydropyran-4-ylethynyl)-1H-indazole C1 (12.0 kg), PdCl₂(PPh₃)₂, (0.26 kg), and CuI (0.35 kg). Reactor A was degassed (vacuum/nitrogen purges×2). To reactor B was charged EtOH (52.1 kg) (to aid in the transfer of trimethyl((tetrahydro-2H-pyran-4-yl)ethynyl)silane), and degassed with (vacuum/nitrogen purges×2). To reactor A was charged trimethyl((tetrahydro-2H-pyran-4-yl)ethynyl)silane (7.42 kg) and EtOH (4.7 kg). To reactor A was charged 45 wt % KOH (9.72 kg) and EtOH (4.6 kg) (to aid in the transfer of the 45 wt % KOH). The agitator was started in Reactor A, the vessel was then degassed (vacuum/nitrogen purges×4), and the contents of Reactor A were heated to 75±5° C. The reaction was held at 76.5 to 77.0° C. for 2 h, and then cooled to 40.1° C. over 20 min. The contents of reactor A were concentrated to a volume of 24 L by vacuum distilled with the maximum temperature of 35.1° C. The contents of reactor A were adjusted to 13.5° C. To a drum was added water (73.9 kg) and concentrated HCl (4.1 kg). The HCl transfer line was rinsed with water (4.7 kg) and charged to the drum. The contents of the drum were mixed (0.5 M HCl soln). The 0.5 M HCl solution (73.9 kg) was transferred to Reactor A over 21 min to cause precipitation of 5-bromo-6-(2-tetrahydropyran-4-ylethynyl)-1H-indazole C2 and a maximum temperature of 20.9° C. (spec. 20±5° C.) during the addition. An aliquot of the slurry was taken and the pH was measured to be 2.0 with a calibrated pH probe. KOH (45 wt %, 0.3 kg) was charged to Reactor A to give a reaction temperature of 15.4° C. An aliquot of the slurry was taken and the pH was measured to be 10.3 with a calibrated pH probe. HCl (0.5 M, 1.2 kg) was transferred over 2 min to reactor A with a maximum temperature of 13.8° C. An aliquot of the slurry was taken and the pH was measured to be 6.03 with a calibrated pH probe. The contents of reactor A were adjusted to 22.1° C. and held for 1 h at 22.1° C. The contents of reactor A were filtered (filtration time 27 min) and washed with water (2×36 kg). The solids were dried on the filter for 50 min, then dried on trays at 50-55° C. for 16 h to afford the product C2.

### Step 2. Synthesis of 5-(4-fluorophenyl)-6-tetrahydropyran-4-yl-H-pyrrolo[2,3-f]indazole (C4)

NaOtBu, 97% (39.2 g, 407.4 mmol, 2.1 equiv.) was added to a reactor. Ethanol (355.2 mL, 6 vols) was added (Note: exothermic reaction) and the mixture was purged with nitrogen. 5-bromo-6-[2-(oxan-4-yl)ethynyl]-1H-indazole C2 (59.2 g, 194 mmol, 1 equiv.) was added at 20° C. to the reactor. 4-fluoroaniline (23.71 g, 20.3 mL, 213.4 mmol, 1.1 equiv.) was then added and the mixture degassed (vacuum and nitrogen purge cycles×3). t-BuXPhos Pd G1 (4.0 g, 5.82 mmol, 0.03 equiv.) at 20° C. was added and the mixture degassed again (vacuum and nitrogen purge cycles×3). The reactor was heated to 65° C. internal temperature for 2 h, then cooled to 60° C. AcOH (55.3 g, 52.8 mL, 921.5 mmol, 4.75 equiv.) at 60° C. was added (Note exothermic reaction, solids precipitate during addition) and the reaction allowed to stir at 60-63° C. for 2 h. The mixture was then cooled to 25° C. Dichloromethane (8 vol) was added to the mixture. 0.5 M NaOH (5 vol) was added and the phases were stirred vigorously for 20 minutes. Additional 0.5 M NaOH was added to adjust the pH to pH 6-7. The phases were separated, and the aqueous phase was separated and extracted with dichloromethane (4 vol). The organic phases were combined, and distilled to ˜3 vol. Additional dichloromethane (6 vol) was added and the distillation to 3 vol. repeated. Addition of dichloromethane, then distillation was repeated until the residual EtOH was reduced to below 1% by NMR. The residual solution of 3 vol dichloromethane was heated to 38° C. Heptane (3 vol) was added and the mixture was stirred for 1 h, then cooled to 20° C. over 3 h. The resulting slurry was filtered and the filter cake washed with 1:1 v/v dichloromethane:heptane. The product was dried under vacuum at 45° C. to afford the product as a white solid (75% yield).

### Step 3. Synthesis of 1-[5-(4-fluorophenyl)-6-tetrahydropyran-4-yl-pyrrolo[2,3-f]indazol-1-yl]-2,2-dimethyl-propan-1-one (C5)

To reactor A under nitrogen was charged 5-(4-fluorophenyl)-6-tetrahydropyran-4-yl-1H-pyrrolo[2,3-f]indazole C4 (8.3 kg) and THE (99.4 kg). The agitator was started in Reactor A. Compound C4 dissolved and the solution was cooled to 1.7° C. KOtBu in THE (15.9 kg) was charged to reactor A over 9 min (temp. range during addition 0.2° C. to 1.6° C.). The transfer line was rinsed with THE (1.0 kg) and transferred to reactor A. The contents of reactor A were stirred for 10 min at 1.6° C. Pivaloyl chloride (3.3 kg) was charged over 32 min to reactor A with the maximum temperature reaching 2.3° C. The transfer line was rinsed with THE (0.5 kg) and transferred to reactor A. The contents of reactor A were held at 0.7° C. to 2.1° C. for 1 h. To a drum was charged NaHCO₃ (2.3 kg) and water (32.0 kg). The contents were briefly mixed to dissolve the NaHCO₃. The contents of reactor A were warmed to 19.0° C. over 2 h 10 min. The NaHCO₃ solution was charged to reactor A over 10 min (max. temp. during addition 19.4° C.). MTBE (29.3 kg) was charged to reactor A. The contents of reactor A were stirred at 25±5° C. for 15 min. The agitator was stopped and the phases separated for 33 min. The aqueous phase was removed. The agitator in reactor A was started. To a drum was added sodium chloride (6.2 kg) and water (26.1 kg). The drum was stirred to give a solution. The brine solution was transferred to reactor A. The contents were stirred for 19 min at 25±5° C. The agitator in reactor A was stopped and the phases settled for 20 min. The aqueous phase was removed. The agitator was started and the organic phase was concentrated by vacuum distillation to 30 L with the maximum distillation temperature of 26.2° C. To reactor A was charged n-heptane (21.9 kg). The contents of reactor A were concentrated to 30 L by vacuum distillation (maximum temperature 25.8° C.). To reactor A was charged n-heptane (21.8 kg) over 17 min. The contents of reactor A were concentrated to 30 L by vacuum distillation (maximum temperature 29.3° C.). To reactor A was charged n-heptane (23.0 kg) over 16 min. The contents of reactor A were stirred at 20±5° C. for 1 h. The slurry was filtered. To reactor A was charged n-heptane (11.2 kg) and transferred to the filter. This was repeated with another n-heptane (11.2 kg) rinse. The cake was dried under nitrogen pressure for 5 h and then loaded into trays and dried for 3 days to afford the product 1-[5-(4-fluorophenyl)-6-tetrahydropyran-4-yl-pyrrolo[2,3-f]indazol-1-yl]-2,2-dimethyl-propan-1-one (C5) as a solvate with THF (5 wt %) by ¹H NMR (6.9 kg, 68%, brown solid).

### Step 4. Synthesis of 1-[5-(4-fluorophenyl)-7-iodo-6-tetrahydropyran-4-yl-pyrrolo[2,3-f]indazol-1-yl]-2,2-dimethyl-propan-1-one (S1)

To reactor A under nitrogen was added 1-[5-(4-fluorophenyl)-6-tetrahydropyran-4-yl-pyrrolo[2,3-f]indazol-1-yl]-2,2-dimethyl-propan-1-one C5 (4.75 kg) and CH₂Cl₂ (29 L). The agitator was started and the jacket was set at −10° C. The solution was cooled to <5.0° C. and N-iodosuccinimide (2.73 kg) was added in three equal portions. At 3.0° C. the 1^{st} portion was added and gave an exotherm to 4.1° C. After 19 min the reaction temperature had cooled to 0.9° C. The 2^{nd} portion was added at 0.9° C. with an exotherm to 2.3° C. After 15 min, the reaction temperature had cooled to 1.4° C. The 3^{rd} portion was added at 1.4° C. with an exotherm to 2.1° C. CH₂Cl₂ (1 L) was charged to reactor A to rinse the N-iodosuccinimide. The jacket temperature was set at 0° C. and the reaction was stirred for 50 min with a final reaction temperature of 3.2° C. To a container was charged sodium thiosulfate pentahydrate (0.85 kg) and water (14.5 L). The contents were mixed to give a solution. The sodium thiosulfate solution (room temperature) was charged in portions to the reaction solution (3.4° C., jacket temperature 0° C.) over 8 min to give an exotherm to 11.6° C. The mixture was warmed to 20° C. stirred for 15 min. The agitator was stopped to let the phases separate for 35 min. The aqueous phase was removed and back extracted with CH₂Cl₂ (5 L). The mixture was stirred 10 min at 20° C. and the agitator was stopped. The phases settled for 10 min and the aqueous phase was removed. The organic phases were combined and charged back to reactor A. The agitator was started. To a container was charged KHCO₃ (0.90 kg) and water (14.1 L). The contents were mixed to give a solution. The KHCO₃ aq. solution was added to reactor A and stirred for 10 min at 20° C. The agitator was stopped and an emulsion had formed. The phases separated overnight and the aqueous phase was removed. The organic phase was charged back to the reactor and rinsed in with CH₂Cl₂ (1 L). A container was charged NaCl (3.0 kg) and potable water (12.0 L). The contents were mixed to dissolve and the brine solution was transferred to reactor A. The contents of reactor A were mixed for 10 min at 20° C. The agitator was stopped and an emulsion had formed. After settling for 2 h the majority of the organic CH₂C2 bottom phase was removed leaving behind about 18 L of emulsion. Water (7.5 L) was added to reactor A with slow stirring (50 rpm) this diluted the brine wash from 20 wt % to approximately 12 wt %. The phases separated in 20 min and the CH₂Cl₂ bottom layer was removed. The organic phase was split in half and concentrated in two flasks. Each flask was concentrated to 5 volumes. To each flask was charged MeOH (10 L) in portions and distilled to 4 volumes. To each flask was charged MeOH (4 L) and distilled to 2 volumes. The contents of each flask were cooled to 0-5° C. and stirred for 1.5 h. Contents of the two flasks were combined into one filter and filtered quickly. The filter cake was washed with 0-10° C. MeOH (2×5 L) and filtered fast. The cake was deliquored for 1 h under vacuum filtration and then loaded into drying trays. The solid was dried overnight at 45° C. in drying trays to afford S4 as a brown solid (5.75 kg, 8.98 wt % solvate).

### Preparation of S3

### 5-(4-fluorophenyl)-7-iodo-1-(phenylsulfonyl)-6-(tetrahydro-2H-pyran-4-yl)-1,5-dihydropyrrolo[2,3-f]indazole (S3)

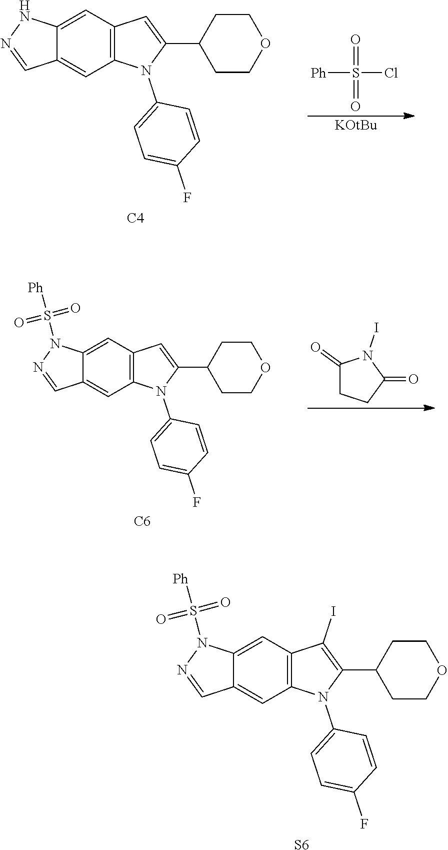

### Step 1. Synthesis of 1-(benzenesulfonyl)-5-(4-fluorophenyl)-6-tetrahydropyran-4-yl-pyrrolo[2,3-f]indazole (C6)

To a solution of 5-(4-fluorophenyl)-6-tetrahydropyran-4-yl-1H-pyrrolo[2,3-f]indazole C6 (10 g, 29.8 mmol) in THE (120 mL) at 0° C. was added KOtBu (4.2 g, 37.3 mmol) and the mixture stirred for 10 min. Benzene sulfonyl chloride (4.4 mL, 34.5 mmol) was added, and the mixture stirred for 1 h at 0° C., then for an additional 1 h at room temperature. The mixture was concentrated in vacuo, and then saturated NH₄Cl and CH₂Cl₂ were added. The organic layer was separated, and dried. Purification by silica gel chromatography (Gradient: 0-60% CH₂Cl₂ in EtOAc) afforded the product as a white solid, containing around 5% of C6 (11.8 g, 83%). ¹H NMR (300 MHz, Chloroform-d) δ 8.38 (t, J=1.0 Hz, 1H), 8.14 (d, J=0.9 Hz, 1H), 8.04-7.93 (m, 2H), 7.57-7.47 (m, 1H), 7.46-7.38 (m, 2H), 7.38-7.30 (m, 3H), 7.15 (t, J=0.9 Hz, 1H), 6.62 (d, J=0.8 Hz, 1H), 4.08-3.94 (m, 2H), 3.37 (td, J=11.8, 2.3 Hz, 2H), 2.82 (ddt, J=11.5, 8.0, 3.9 Hz, 1H), 1.98-1.70 (m, 5H). LCMS m/z 476.2 [M+H]*.

### Step 2. Synthesis of 1-(benzenesulfonyl)-5-(4-fluorophenyl)-7-iodo-6-tetrahydropyran-4-yl-pyrrolo[2,3-f]indazole (S3)

To a solution of 1-(benzenesulfonyl)-5-(4-fluorophenyl)-6-tetrahydropyran-4-yl-pyrrolo[2,3-f]indazole C6 (151.8 g, 319.2 mmol) in CH₂Cl₂ (1.52 L) cooled to 0° C. was added 1-iodopyrrolidine-2,5-dione (74.5 g, 321.2 mmol), in 4 approximately equal portions over 45 min, additions were 15 min apart. After each addition, a slight exotherm was observed, the internal temp. rose to ˜2° C. The reaction mixture was warmed to room temperature and stirred overnight. CH₂Cl₂ (500 mL) was added, and the reaction was stirred for 15 min. Water (1 L) was added, followed by 1 M aqueous sodium thiosulfate (200 mL). The mixture was stirred for 20 min, then the organic layer was separated, and the aqueous layer was extracted with CH₂Cl₂ (50 mL). Combined organic layers were washed successively with water, saturated aqueous sodium bicarbonate, and brine (1.5 L each). The organic layer was then dried (MgSO₄), filtered and concentrated to afford a solid residue. The residue was treated with MTBE (500 mL), then stirred for 90 min. The resulting solid was isolated via filtration, washing with MTBE (2×200 mL) and dried under suction for 30 min. The solid was further dried under vacuum (2 mbar, 75° C.) for 30 min, to afford the product as pale, cream-colored crystals. 1-(benzenesulfonyl)-5-(4-fluorophenyl)-7-iodo-6-tetrahydropyran-4-yl-pyrrolo[2,3-f]indazole (181.4 g, 94%). ¹H NMR (400 MHz, DMSO-d₆) δ 8.51 (d, J=0.9 Hz, 1H), 8.06 (t, J=0.9 Hz, 1H), 7.87-7.80 (m, 2H), 7.71-7.63 (m, 1H), 7.62-7.45 (m, 6H), 7.25 (d, J=1.0 Hz, 1H), 3.96-3.85 (m, 2H), 3.22 (td, J=11.8, 1.9 Hz, 2H), 2.93 (tt, J=12.4, 3.6 Hz, 1H), 2.29 (qd, J=12.6, 4.4 Hz, 2H), 1.63 (dd, J=13.5, 3.5 Hz, 2H). 19F NMR (376 MHz, DMSO-d₆) δ −111.78. LCMS m/z 602.1 [M+H]⁺.

### Preparation of Compound I

### 4-[5-(4-fluorophenyl)-6-tetrahydropyran-4-yl-1H-pyrrolo[2,3-f]indazol-7-yl]benzoic acid (Compound I)

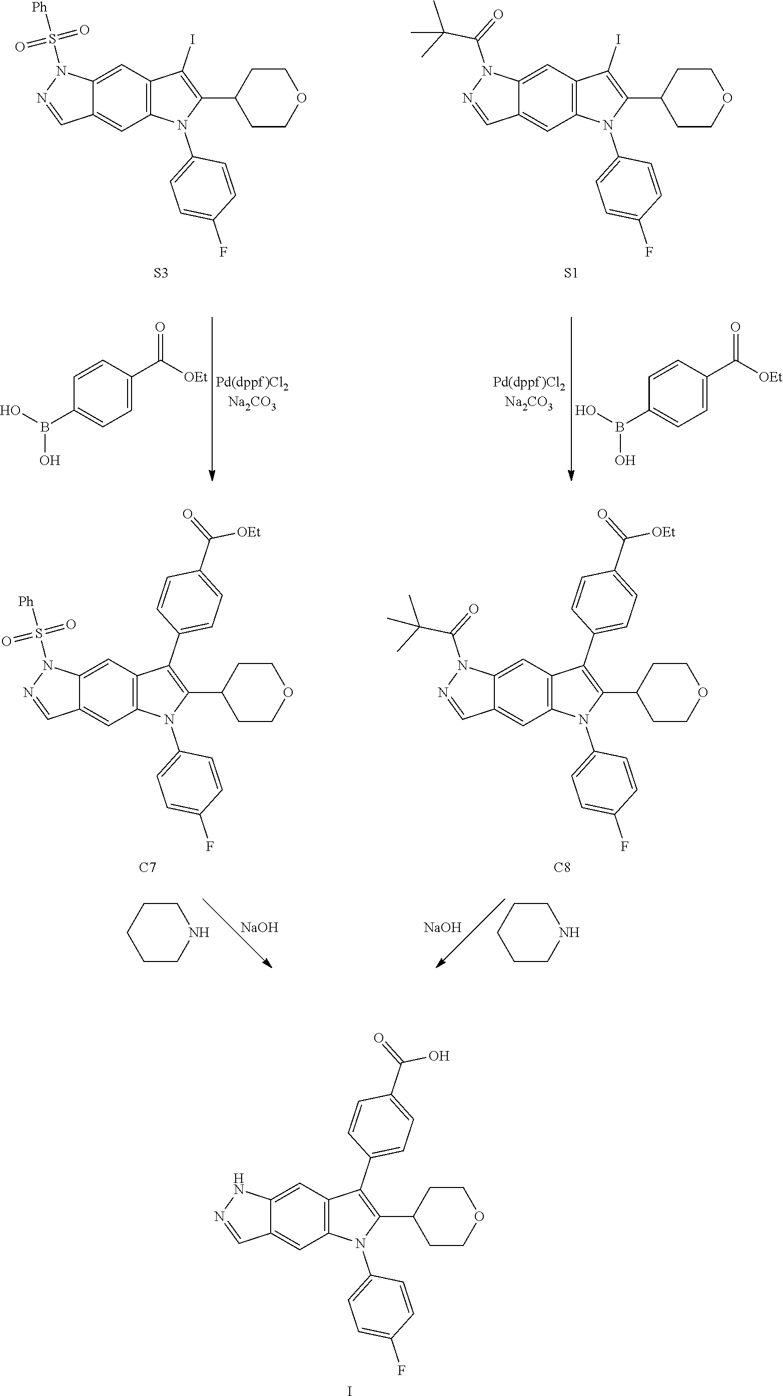

### Preparation of 4-[5-(4-fluorophenyl)-6-tetrahydropyran-4-yl-1H-pyrrolo[2,3-f]indazol-7-yl]benzoic acid (33 (Compound I)) from S3

### Step 1. Synthesis of ethyl 4-[1-(benzenesulfonyl)-5-(4-fluorophenyl)-6-tetrahydropyran-4-yl-pyrrolo[2,3-f]indazol-7-yl]benzoate (C7)

A mixture of 1-(benzenesulfonyl)-5-(4-fluorophenyl)-7-iodo-6-tetrahydropyran-4-yl-pyrrolo[2,3-f]indazole S3 (103.8 g, 172.6 mmol), (4-ethoxycarbonylphenyl)boronic acid (67 g, 345.4 mmol), Pd(dppf)Cl₂ (6.4 g, 7.8 mmol) and Na₂CO₃ (270 mL of 2 M, 540 mmol) in 1,4-dioxane (1 L) was purged with nitrogen for 20 min, then heated at 90° C. for 1 h. The mixture was filtered through Celite®, washing with EtOAc (500 mL). The filtrate was concentrated to dryness in vacuo. EtOAc (1 L) and water (300 mL) were added. The organic layer was separated and filtered through Celite®. The organic layer was then washed with 1 M NaOH (300 mL×2), and brine. The organic layer was dried, and concentrated in vacuo. The residue was dissolved in CH₂Cl₂(200 mL) and the solution was purified by silica gel chromatography. (Column: 3 kg Silica gel. Gradient: 0-100% EtOAc in heptane) to afford the product as a white, foamy solid (˜102 g). TBME (550 mL) was added, and the suspension was allowed to stir at room temperature for 1 h. The solid was filtered (washing with 200 mL MTBE). CH₂Cl₂ (300 mL) and EtOAc (400 mL) were added to afford a clear solution which was treated with MP-TMT Pd resin (45 g) and allowed to stir overnight. The suspension was filtered, and the filtrate concentrated in vacuo to afford the product as a white solid (96 g, 89%). ¹H NMR (300 MHz, Chloroform-d) δ 8.33-8.22 (m, 2H), 8.15 (d, J=0.8 Hz, 1H), 8.10 (t, J=0.9 Hz, 1H), 7.91 (dd, J=8.4, 1.3 Hz, 2H), 7.65-7.56 (m, 2H), 7.56-7.46 (m, 1H), 7.46-7.35 (m, 4H), 7.35-7.23 (m, 2H), 7.06 (d, J=1.0 Hz, 1H), 4.49 (q, J=7.1 Hz, 2H), 3.86 (dd, J=11.4, 3.5 Hz, 2H), 3.22 (t, J=11.0 Hz, 2H), 3.05 (ddd, J=12.2, 8.9, 3.3 Hz, 1H), 1.83 (qd, J=12.6, 4.3 Hz, 2H), 1.64 (s, 2H), 1.49 (t, J=7.1 Hz, 3H). LCMS m/z 624.3 [M+H]*.

### Step 2. 4-[5-(4-fluorophenyl)-6-tetrahydropyran-4-yl-1H-pyrrolo[2,3-f]indazol-7-yl]benzoic acid (Compound I)

Piperidine (54 mL, 546.0 mmol) and NaOH (1350 mL of 1 M, 1.350 mol) were added to a solution of ethyl 4-[1-(benzenesulfonyl)-5-(4-fluorophenyl)-6-tetrahydropyran-4-yl-pyrrolo[2,3-f]indazol-7-yl]benzoate C7 (170 g, 272.6 mmol) in THE (1800 mL) and MeOH (1800 mL) and the mixture was heated to 50° C. for 3.5 h. Upon cooling, HCl (700 mL of 2 M, 1.40 mol) was added to adjust the mixture to pH=2. The solvent volume was reduced (by 3 L) by concentration in vacuo. The light yellow precipitate was filtered off, washing the filter cake with water (×3), TBME (250 mL×2) and EtOAc (250 mL×2). The solid filter cake was dried under vacuum. The solid was then dissolved in EtOAc (1.2 L) and the solution heated to reflux for 10 min. ˜600 mL of solvent was removed by concentration under vacuum. An additional 600 mL of EtOAc was added and the process of refluxing for 10 min followed by removal of 1 L of solvent was repeated. Finally, EtOAc (1 L) was added and the mixture was heated at reflux for 2 h. Upon cooling overnight, the resulting solid was filtered off, washing with EtOAc (1×). This solid was then dried under vacuum at 60° C. for 4 h affording the product as a white solid (97.4 g, 78%). ¹H NMR (400 MHz, DMSO-d₆) δ 13.01 (s, 1H), 12.61 (s, 1H), 8.17-8.05 (m, 2H), 8.01 (d, J=1.0 Hz, 1H), 7.69-7.58 (m, 4H), 7.57-7.45 (m, 2H), 7.31-7.23 (m, 1H), 7.08 (d, J=1.1 Hz, 1H), 3.73 (dt, J=11.2, 3.1 Hz, 2H), 3.20-2.92 (m, 3H), 1.66 (h, J=4.2 Hz, 4H). LCMS m/z 456.0 [M+H]⁺.

### Preparation of 4-[5-(4-fluorophenyl)-6-tetrahydropyran-4-yl-1H-pyrrolo[2,3-f]indazol-7-yl]benzoic acid (Compound I) from S1

### Step 1. Synthesis of ethyl 4-[1-(2,2-dimethylpropanoyl)-5-(4-fluorophenyl)-6-tetrahydropyran-4-yl-pyrrolo[2,3-f]indazol-7-yl]benzoate (C8)

A mixture of 1-[5-(4-fluorophenyl)-7-iodo-6-tetrahydropyran-4-yl-pyrrolo[2,3-f]indazol-1-yl]-2,2-dimethyl-propan-1-one S1 (1.0 g, 1.83 mmol), (4-ethoxycarbonylphenyl)boronic acid (556.9 mg, 2.87 mmol), and Pd(dppf)Cl₂ (76.3 mg, 0.09 mmol) was placed under a nitrogen atmosphere. 1,4-dioxane (8.8 mL) and sodium carbonate (3.2 mL of 2 M, 6.4 mmol) were added and the mixture was heated at 90° C. for 30 min. Purification by silica gel chromatography (0-5% EtOAc in CH₂Cl₂) gave a light tan solid. Minimal Et₂O and heptane were added to the solid, and the white solid precipitate was filtered off. The solid was dissolved in dichloromethane (ca. 25 mL). MP-TMT resin (1.1 g) was added and the mixture stirred for 1 h at room temperature. The resin was filtered off and the filtrate concentrated in vacuo to afford the product as a white solid (681.7 mg, 62%). ¹H NMR (400 MHz, Chloroform-d) δ 8.45 (s, 1H), 8.21 (d, J=7.8 Hz, 2H), 8.08 (s, 1H), 7.58 (d, J=8.0 Hz, 2H), 7.46 (dd, J=8.0, 4.9 Hz, 2H), 7.35 (t, J=8.2 Hz, 2H), 7.12 (s, 1H), 4.48 (q, J=6.9 Hz, 2H), 3.86 (dd, J=11.3, 4.2 Hz, 2H), 3.23 (t, J=11.7 Hz, 2H), 3.09-2.99 (m, 1H), 1.90-1.77 (m, 2H), 1.64 (d, J=13.2 Hz, 2H), 1.58 (s, 9H), 1.48 (t, J=7.1 Hz, 3H). LCMS m/z 568.5 [M+H]⁺.

### Step 2. Synthesis of 4-[5-(4-fluorophenyl)-6-tetrahydropyran-4-yl-1H-pyrrolo[2,3-f]indazol-7-yl]benzoic acid (Compound I)

NaOH (6 mL of 1 M, 6.0 mmol) and piperidine (260 μL, 2.629 mmol) were added to a solution of ethyl 4-[1-(2,2-dimethylpropanoyl)-5-(4-fluorophenyl)-6-tetrahydropyran-4-yl-pyrrolo[2,3-f]indazol-7-yl]benzoate C8 (682 mg, 1.20 mmol) in THE (14 mL) and MeOH (7 mL). The mixture was heated at 50° C. for 1 h. The solvent was concentrated, and the residue re-dissolved in minimal water. HCl (6 mL of 1 M, 6.0 mmol) was added and a precipitate formed. The solid was filtered off and washed with excess water to afford the product as an off-white solid. (455.7 mg, 83%). ¹H NMR (400 MHz, DMSO-d₆) δ 13.02 (s, 1H), 12.60 (s, 1H), 8.11 (d, J=7.7 Hz, 2H), 8.00 (s, 1H), 7.63 (t, J=7.3 Hz, 4H), 7.51 (t, J=8.4 Hz, 2H), 7.26 (s, 1H), 7.07 (s, 1H), 3.73 (d, J=11.2 Hz, 2H), 3.15-3.07 (m, 2H), 3.05-2.96 (m, 1H), 1.72-1.61 (m, 4H). LCMS m/z 456.4 [M+H]⁺.

### Alternative Preparation of 4-[5-(4-fluorophenyl)-6-tetrahydropyran-4-yl-1H-pyrrolo[2,3-f]indazol-7-yl]benzoic acid (Compound I) from SI

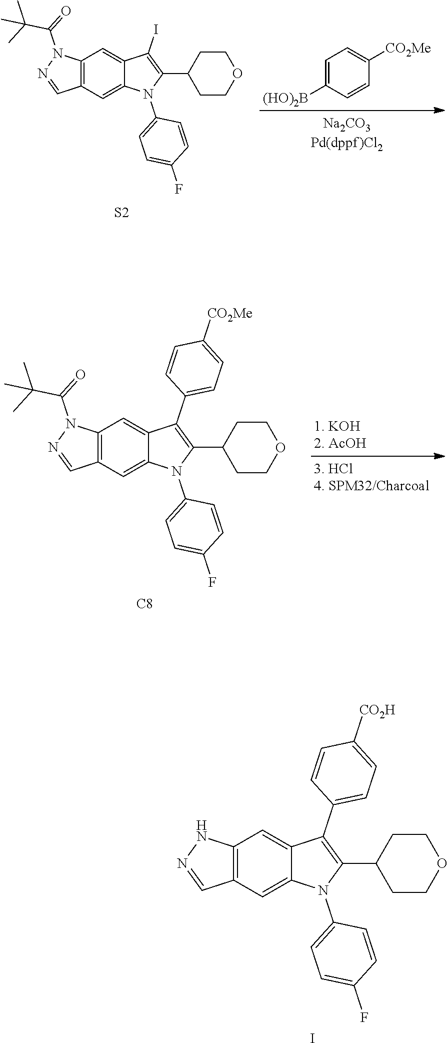

### Step 1. Synthesis of ethyl 4-[1-(2,2-dimethylpropanoyl)-5-(4-fluorophenyl)-6-tetrahydropyran-4-yl-pyrrolo[2,3-f]indazol-7-yl]benzoate (C8)

To reactor A under nitrogen was added S1 (5.42 kg), 4-methoxycarbonyl benzene boronic acid (1.786 kg), Na₂CO₃ (2.986 kg), 1,4-Dioxane (36 L), and potable water (12.5 L). The agitator was started and reactor A was degassed with one vacuum/nitrogen cycle. Nitrogen was bubbled via the bottom of the reaction mixture with stirring at room temperature while venting the nitrogen via the top of the reactor for 1 h. Pd(dppf)Cl₂—CH₂Cl₂ adduct (0.186 kg) was charged as a solid to reactor A. 1,4-Dioxane (1 L) was degassed (nitrogen bubbling for 5 min), and used to rinse the solids off the walls of reactor A. Reactor A was heated to 74° C.-78° C. for 3.5 h. The reaction was then held at 20° C. overnight, and then heated to 38.1° C. Potable water (24 L) was added to reactor A over 18 min, while maintaining the temperature at 36.0° C. to 38.1° C. The slurry was cooled to 20° C. over 2.5 h and filtered (filtration time 25 min). The cake was washed with potable water (2 L×2) and then was deliquored overnight. The wet filter cake solid and CH₂Cl₂ (25 L) was charged to reactor A. To a container was charged NaCl (1.1 kg) and potable water (9.9 kg). The contents were mixed to dissolve the NaCl. The brine solution was charged to reactor A. The agitator was started and the contents of reactor A were mixed at 22° C. for 15 min. The agitator was stopped and the layers separated for 22 min. The organic layer was removed (no emulsion). The aqueous layer was back extracted by charging CH₂Cl₂ (5 L) to reactor A. The agitator was started and mixed for 15 min. The agitator was stopped and the phases settled for 15 min. The CH₂Cl₂ layer was removed and combined with the 1^{st} CH₂Cl₂ layer. To reactor B was charged charcoal (1 kg) and the solution of product C8 in CH₂Cl₂. The agitator was started and stirred at room temperature for 23.5 h. A filter was set with Celite® plug and the contents of reactor B were filtered via the Celite® filter. The Celite® cake was washed with CH₂Cl₂ (6 L). The CH₂Cl₂ solution was concentrated to 2.5 volumes by vacuum distillation in two separate flasks. Heptanes (7 L) were charged to each flask while rotating, causing the formation of a thick slurry. Both flasks were held at room temperature overnight, and concentrated to 4 volumes. Each flask was cooled to 0-5° C., and rotated for 1 h. The contents of each flask were combined and filtered. The cake was washed with a CH₂Cl₂:heptanes (1:5) solution. The solids were loaded into trays and dried at 50° C. in a vacuum oven for 3 days, to afford the product C8 as a brown solid (5.3 kg, 88% yield, 8.0 wt % 1,4-dioxane solvate).

### Step 2. Synthesis of 4-[5-(4-fluorophenyl)-6-tetrahydropyran-4-yl-1H-pyrrolo[2,3-f]indazol-7-yl]benzoic acid (Compound I)

Part A. Hydrolysis

To reactor A under nitrogen was added ethyl 4-[1-(2,2-dimethylpropanoyl)-5-(4-fluorophenyl)-6-tetrahydropyran-4-yl-pyrrolo[2,3-f]indazol-7-yl]benzoate (C8) (5.2 kg), ethanol (26 L, 5 vol.), water (14.3 L, 2.7 equiv.), and 45% KOH (6.12 kg, 49.1 mol, 5.2 equiv.). The agitator was started and the reaction mixture was heated to 70-75° C. for 1 h. The reaction was cooled to room temperature and filtered via a plug of Celite®. Reactor A was rinsed with ethanol (5 L, 1 vol.) and used to rinse the Celite®. To reactor A was added acetic acid (2.968 kg, 49.5 mol, 5.2 equiv.) and water 17 L, 3.3 vol.). The acetic acid/water was heated to 46° C. and stirred at 200 rpm. The solution of C8 in ethanol was added over 22 min to the acetic acid/water to give a fine slurry. The temperature was 46.3° C. and the pH was 6.36. Acetic acid (1.176 kg, 19.7 mol, 2 equiv.) was added and the pH was 5.86 measured with a pH probe. The jacket was set with the following profile to hold at 50° C. for 9 h, cool to 20° C., and hold at 20° C. overnight. The slurry was stirred at 20° C. for 6 h before filtering. The slurry was filtered for 24 h. Water was charged to wash the cake (16 L, 3 vol.), which was filtered for an additional day to afford Compound I as a potassium salt (brown solid, approximately 80% yield).

Part B. Free Acid Formation

To reactor A was added the wet 4-[5-(4-fluorophenyl)-6-tetrahydropyran-4-yl-1H-pyrrolo[2,3-f]indazol-7-yl]benzoic acid (Compound I) potassium salt (3.4 kg). Potable water (44 L) was added to reactor A and the agitator was started. The mixture was stirred slowly at first and then at 133 rpm to give a nice slurry. 1M HCl (7.4 L) (0.1 equivalents excess based on an 80% isolated yield of the potassium salt of Compound I) was charged to reactor A. Stirring was maintained for 3 h at 25° C., and then left overnight. The mixture was filtered on two filters by splitting the batch in half. After filtering for 8 h, the cake was washed with potable water (2 L) for each filter. The filtering continued overnight, and the cake was dried with vacuum filtration for 20 h. Compound I was dried under vacuum for 2 days at 50° C. and then for 2 days at 30° C. to afford the product (free acid) as a brown solid (3.4 kg, 80% yield).

Part C. Palladium Scavenging

To reactor A under nitrogen was charged Compound I (3.4 kg, 7.47 mol), MeTHF (34 L), PhosphonicsS SPM32 (0.686 kg) (PhosphonicsS SPM32=3-Mercaptopropyl ethyl sulfide Silica, metal scavenging functionalized silica), and carbon (0.682 kg). The mixture was heated to 68° C. for 17 h with stirring. The mixture was cooled to 43° C. and filtered via a filter lined with a 2 inch silica gel pad. The silica was rinsed with MeTHF (6 L). A 2^{nd} treatment was carried out by charging SPM32 (0.68 kg), carbon (0.681 kg), and the filtrate of Compound I in MeTHF to a 100 L reactor under nitrogen. MeTHF (4 L) was used to aid in the transfer of the solution of Compound I in MeTHF back to the reactor. The stirring was initiated and the mixture was heated to 68° C. The mixture was stirred for 23 h, cooled to 50-60° C., and filtered as described above. This process was repeated two additional times. The filtrate was filtered via a 0.2 micron filter into a rotovap flask and concentrated to a wet solid. EtOH (8 L) was added and the vacuum distillation was continued to afford a solid. The solid was dried under vacuum at 50° C. overnight to afford Compound I (1.95 kg, 8% ethanol solvate).

Part D. Drying Procedure

To a flask containing Compound I (1.95 kg, 8 wt % ethanol solvate) was added anhydrous CH₂Cl₂ (10 L). The mixture was distilled under vacuum to viscous slurry. CH₂Cl₂ (10 L) was added and the mixture was distilled under vacuum again, to give a wet solid. CH₂Cl₂ (10 L) was added to afford a slurry. The slurry was transferred to reactor A and additional CH₂Cl₂ (10 L) was used to transfer the residual contents of the flask to reactor A. The agitator was started, and the slurry was heated to 37° C., and held for 2 h at 35-37° C. The slurry was then cooled to 18° C. over 30 min, and held at 18° C. for 30 min. The slurry was filtered and washed with CH₂Cl₂ (2 L×2) at room temperature over 2 h. The filtered solid material was loaded into trays and dried in a vacuum oven at 70° C. overnight. The solids were broke apart into a fine powder, and dried for an additional 4 h to afford Compound I as a beige solid (1.36 kg, 72% yield, corrected for EtOH solvate, and 0.4% water).

### Example 2: Preparation of a Coated Tablet Containing 250 mg of Compound I

The following materials listed in Table 3 can be used in this exemplary preparation of a tablet containing 250 mg of Compound I.

TABLE 3
Materials in Exemplary Preparation of Table Containing 250 mg of Compound I.

| | % W/W Core | Tablet Quantity | Batch Quantity |
|---|---|---|---|
| Material | Tablet | (mg) | (kg) |
| Compound I (in spray-dried dispersion | 38.83 | 250.0 | 1.200 |
| with hypromellose acetate succinate) | | | |
| Hypromellose acetate succinate (in SDD) | 9.71 | 62.5 | 0.30 |
| Microcrystalline cellulose, NF Avicel PH- | 15.78 | 101.56 | 0.4876 |
| 101 (intra-granular) | | | |
| Lactose Monohydrate FastFlo 316 | 15.78 | 101.56 | 0.4876 |
| Croscarmellose sodium Ac Di-Sol, NF | 2.91 | 18.75 | 0.090 |
| (intra-granular) | | | |
| Sodium stearyl fumarate, NF (intra- | 1.94 | 12.50 | 0.060 |
| granular) | | | |
| Microcrystalline cellulose, NF Avicel PH- | 9.71 | 62.50 | 0.300 |
| 200 (extra-granular) | | | |
| Croscarmellose sodium Ac Di-Sol, NF | 1.46 | 9.38 | 0.045 |
| (extra-granular) | | | |
| Sodium stearyl fumarate, NF (extra- | 0.97 | 6.25 | 0.030 |
| granular) | | | |
| Nonfunctional Film Coating | 2.91 | 18.75 | 0.090 |
| TOTAL | 100 | 643.75 | 3.09 |

In this exemplary preparation, the spray-dried dispersion comprising Compound I and hypromellose acetate succinate, microcrystalline cellulose, lactose monohydrate, and croscarmellose sodium can be sieved, combined in a bin blender, and blended. Sieved sodium stearyl fumarate can be added to the bin blender, and the mixture can be blended. The mixture can be then dry granulated and milled to form milled granules. These milled granules can be added to a bin blender, to which can be added sieved microcrystalline cellulose and sieved croscarmellose sodium. The mixture can be blended. Sieved sodium stearyl fumarate can be added to the bin blender, and the mixture can be blended. The resulting blend can be discharged and then charged to a tablet press. The blend can be compressed into tablets, which can be discharged. The non-functional film coating can be applied to the tablet comprising Compound I using traditional tablet film coating processes.

### Example 3: Safety and Efficacy Study of Compound I

### Phase 1

A randomized, double-blinded, placebo-controlled single and multiple-dose Phase I study evaluating Compound I has been completed in healthy subjects. This study demonstrated that single and multiple doses of Compound I were safe and well-tolerated in healthy subjects. There were no serious adverse events.

### Phase 2

Compound I will be administered in a randomized, double-blind, placebo-controlled Phase 2 study.

Study Design:

In this Phase 2 study, approximately 40 subjects with the PiZZ genotype and antigenic AAT levels <8 μM at screening will be randomized to receive Compound I or placebo. The first 20 subjects will be randomized (2:2:1) to Compound 1500 mg g12 h (n=8), Compound I 300 mg q12 h (n=8), or placebo (n=4). The remaining 20 subjects will be randomized (2:2:1) to one of two Compound I groups (planned doses of 500 mg q12 h (n=8) and 100 mg q12 h (n=8)) or placebo (n=4). The final doses of Compound I may be changed for the second group of 20 subjects based on ongoing review of available pharmacokinetics and safety data. Randomization will be stratified by percent predicted forced expiratory volume in 1 second (ppFEV₁) obtained either during the Screening Period or from a historical ppFEV₁ value (<50% versus ≥50%).

Study Duration:

Excluding the Screening Period, each subject will participate in the study for approximately 56 days: 28 days for the Treatment Period and 28 days for the Safety Follow-up Period.

Strength and Route of Administration of Investigational Drug and Placebo:

100 mg and 250 mg tablets and matching placebo for oral administration.

Inclusion Criteria will include
1. Subjects will be 18 through 80 years of age, and females will have a negative pregnancy test at screening and Day 1.
2. Subjects will have a PiZZ genotype.
3. Plasma antigenic AAT level<8 μm (if applicable, as determined at least 42 days after last dose of augmentation therapy).

Exclusion Criteria will include
1. Subjects meeting any of the following criteria:
   Subjects who have undergone solid organ, lung, or hematological transplantation or are currently on a transplant list.
   Subjects who have undergone gastrectomy or other gastrointestinal tract surgery, except appendectomy, cholecystectomy, and hemorrhoid surgery.
   Subjects who have cancer, except for squamous cell skin cancer, basal cell skin cancer, Stage 0 cervical carcinoma in situ, and stage 0 or 1 melanoma (all 4 with no recurrence during the last 5 years).
2. Subjects who have a history of use of gene therapy or RNAi therapy.
3. Subjects who have used oral corticosteroids (at any dose) for a duration of greater than 3 months within the 3 months before screening.
4. Subjects wo have had illegal drug use within 1 year before screening as deemed by the investigator, including but not limited to cocaine, heroin, and other opioids.
5. Spirometry will be performed post-bronchodilator and according to the American Thoracic Society Guidelines/European Respiratory Society Guidelines. If spirometry cannot be performed, historical FEV₁ results within 1 year before screening can be used to determine eligibility. A post-bronchodilator forced expiratory volume in 1 second (FEV₁) value<30% of predicted mean for age, sex, and height (equations of the Global Lung Function Initiative [GLI]) during screening.
6. Subjects who have all clinically important pulmonary disease other than AATD-related COPD (including but not limited to physician-diagnosed COPD not related to AATD, interstitial lung disease, cystic fibrosis, pulmonary hypertension with or without cor pulmonale, history of pulmonary embolism, or malignant lung cancer) or unstable AATD-related COPD.
7. Subjects who have a documented chronic need for positive airway pressure therapy beyond nocturnal use.
8. Subjects who have a history of chronic liver disease or a history of clinically important liver disease within the previous 12 months before screening.
9. Subjects who have documented medical history or diagnosis of clinically evident liver disease, including but not limited to a prior diagnosis of hepatitis of any etiology, cirrhosis, portal hypertension, or confirmed or suspected esophageal varices.
10. Subjects who have any of the following abnormal laboratory values at screening:
   Platelet count<150×10⁹/L
   Albumin≤3.5 g/dL
   International normalized ratio≥1.2
   Hemoglobin≤10 g/dL
   Total bilirubin≥upper limit of normal (ULN)
   Aspartate transaminase (AST), alanine transaminase (ALT), gamma-glutamyl transferase (GGT), or alkaline phosphatase (ALP)>2×ULN
   Estimated glomerular filtration rate≤30 mL/min/1.73 m² (calculated by the Modification of Diet in Renal Disease Study Equation)
11. Subjects who have risk factors for Torsade de Pointes or concomitant medications that prolong the QT/QTc interval or any history of cardiac disorders.
12. Subjects who show any clinically significant ECG abnormality or median QTcF of triplicate standard 12-lead ECGs>450 msec at screening.
13. Subjects who have a history of Gilbert's Syndrome.
14. Subjects who are positive for HBsAg, HCV antibody and RNA, or HIV-1 and HIV-2 antibodies during screening.
15. Subjects having hypersensitivity to any component of the investigational drug product or placebo (e.g., lactose).
16. Subjects for whom discontinuation of augmentation therapy is not considered to be in their best interest, based on the clinical judgement of the treating physician.

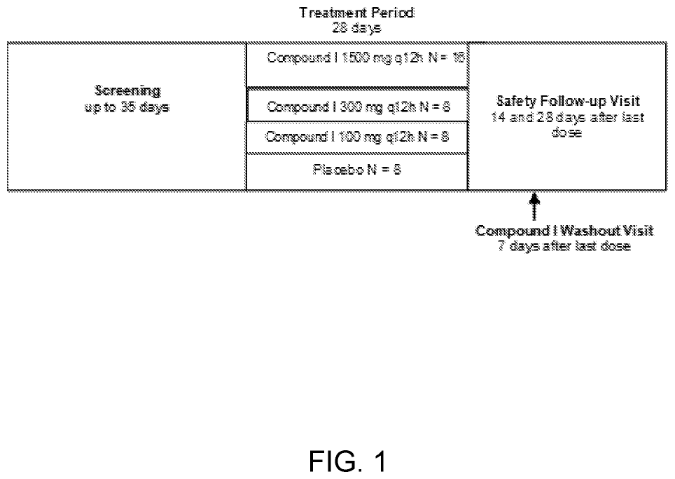

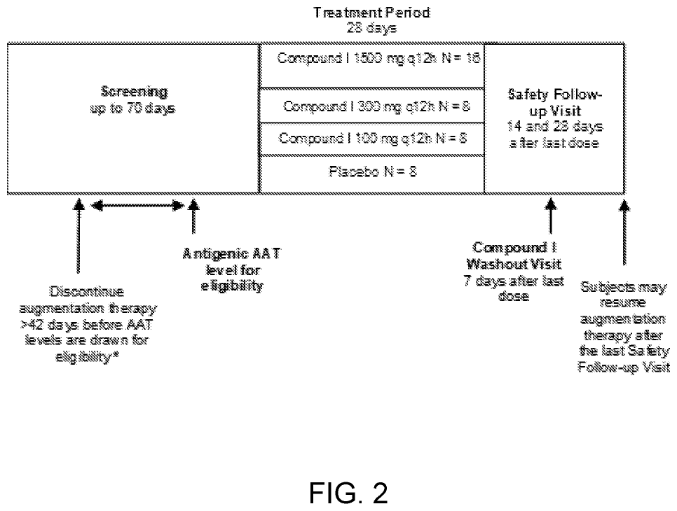

Schematics of the study design are shown in FIGS. 1 and 2, which are not drawn to scale and reflect the overall planned randomization. In FIGS. 1 and 2, “N” refers to the number of subjects, and “q12 h” means “every 12 hours.” Neither figure is drawn to scale, and both reflect the overall planned randomization. Subject numbers in FIGS. 1 and 2 include subjects who have never been on augmentation therapy and subjects who have been on augmentation therapy at any time.

For subjects who have never been on augmentation therapy, antigenic AAT levels must be drawn to confirm eligibility and sent to the central laboratory; results must be obtained and confirmed to be less than 8 μM before randomization. Once antigenic AAT levels have been confirmed to meet this eligibility criterion, randomization and Day 1 can occur any time within the remaining screening window. Sites should allow at least 14 days for sample processing and antigenic AAT level result reporting.

Subjects who have been on augmentation therapy at any time must discontinue augmentation therapy more than 42 days before antigenic AAT levels are drawn and sent to the central laboratory to confirm eligibility; results must be confirmed to be less than 8 μM before randomization. Once antigenic AAT levels have been confirmed to meet this eligibility criterion, randomization and Day 1 can occur any time within the remaining screening window. Sites should allow at least 14 days for sample processing and antigenic AAT level results reporting. Subjects can resume augmentation therapy after completion of assessments at the last Safety Follow-up Visit. Blood samples will be obtained for antigenic and functional AAT levels at the same time that the other screening laboratory assessments are performed. If the subject received the last dose of augmentation therapy more than 42 days prior, this sample can be used to measure antigenic AAT level for eligibility. If samples are obtained less than or equal to 42 days after the last dose of augmentation therapy, another sample must be drawn more than 42 days after the last dose of augmentation therapy and sent to the central laboratory to confirm eligibility.

As depicted in FIGS. 1 and 2, the study will include a screening period, a treatment period, a washout visit, and a follow-up visit. As described above, excluding the screening period, each subject will participate in the study for approximately 56 days: 28 days for the Treatment Period and 28 days for the Safety Follow-up Period. Assuming that 10% of the randomized subjects have a missing value at Day 28, the sample size provides adequate precision to estimate the absolute plasma functional AAT levels at Day 28 for the Compound I500 mg q12 h group. In addition, a sample size of 16 provides adequate precision to estimate the plasma functional AAT levels at Day 28 for a given dose group.

For subjects who have never been on augmentation therapy, the Screening Period (Day −35 through Day −1) will occur within 35 days before the first dose of Compound I.

For subjects who have been on augmentation therapy at anytime, the Screening Period (Day −70 through Day −1) will occur up to 70 days before the first dose of Compound I. The last dose of augmentation therapy must be given at least 42 days before Day 1. To establish eligibility, an antigenic AAT level must be drawn (and results reviewed to confirm eligibility) at least 42 days after the last dose of augmentation therapy. Subjects will remain off augmentation therapy thereafter until after the Safety Follow Visit has been conducted. Subjects must discontinue augmentation therapy at least 42 days before the first dose of study drug. Subjects can resume augmentation therapy after completion of assessments at the last Safety Follow-up Visit.

As noted above, the study population will be comprised of male and female subjects with a diagnosis of COPD and AATD with a confirmed PiZZ genotype. In Part A, a total of 3 doses of Compound I will be evaluated: 500 mg q12 h, 300 mg q12 h, and 100 mg g12 h. Compound I will be administered orally, 2 times a day, approximately 12 hours apart (2 hours), under fasted conditions, wherein subjects will abstain from all food and drink (except water) at least 2 hours before and 2 hours after morning and evening dose of study drug on all study days.

The primary endpoint to assess efficacy is the change from baseline in plasma functional AAT levels at Day 28. The primary comparison consists of pairwise comparison between a dose of Compound I and placebo that achieves 90% power on the primary endpoint. As used herein, “baseline value” will be the most recent non-missing measurement (scheduled or unscheduled) collected before the first dose of study drug. For ECGs, the baseline value will be defined as the average of the non-missing pretreatment measurements (triplicate) before the first dose of Compound I. As used herein, “change (absolute change) from baseline” will be calculated as Post-baseline value−Baseline value. As used herein, “relative change from baseline” will be calculated and expressed in percentage as 100%×(post-baseline value−Baseline value)/Baseline value. The primary analysis will be based on a mixed-effects model for repeated measures (MMRM) with change from baseline at Days 7, 14 and 28 as the dependent variable.

Plasma samples will be collected to evaluate the effect of Compound I on AAT function and antigenic levels in subjects with the PiZZ genotype based on the mechanism of action of Compound I. All safety and PK assessments to be performed are standard measurements for clinical studies in drug development.

The overall safety and tolerability assessments of Compound I will be assessed in terms of endpoints, including:
Incidence of treatment emergent adverse events (TEAEs)
Clinical laboratory values (i.e., hematology, serum chemistry, coagulation, and urinalysis)
Standard 12-lead ECGs
Vital signs
Pulse oximetry

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

1. A method of treating alpha-1 antitrypsin deficiency comprising administering to a patient in need thereof Compound I: 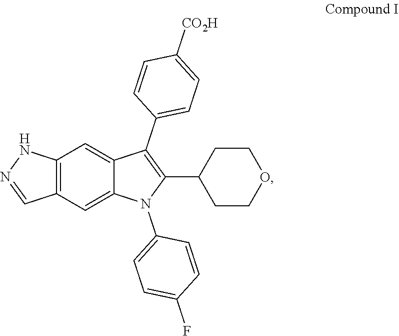 [SMILES: O=C(O)c1ccc(-c2c(C3CCOCC3)n(-c3ccc(F)cc3)c3cc4cn[nH]c4cc23)cc1]
a deuterated derivative thereof, and/or a pharmaceutically acceptable salt thereof in a daily amount of 250 mg to 2500 mg.

2. The method according to claim 1, wherein the patient has the PiZZ genotype.

3. The method according to claim 1, wherein the patient has an SZ mutation in alpha-1 antitrypsin.

4. The method according to claim 1, wherein Compound I, a deuterated derivative thereof, and/or a pharmaceutically acceptable salt thereof is administered in a daily amount of 200 mg, 250 mg, 500 mg, 600 mg, 750 mg, 1000 mg, 1250 mg, 1500 mg, 1750 mg, 2000 mg, or 2500 mg.

5. The method according to claim 1, wherein Compound I, a deuterated derivative thereof, and/or a pharmaceutically acceptable salt thereof is administered in a daily amount of 200 mg, 600 mg, or 1000 mg.

6. The method according to claim 1, wherein Compound I, a deuterated derivative thereof, and/or a pharmaceutically acceptable salt thereof is administered once daily or multiple times daily.

7. The method according to claim 1, wherein Compound I, a deuterated derivative thereof, and/or a pharmaceutically acceptable salt thereof is administered every 8 hours (q8 h) or every 12 hours (q12 h).

8. The method according to claim 1, wherein 100 mg, 250 mg, 300 mg, 500 mg, 750 mg, 1000 mg, 1250 mg, or 1500 mg of Compound I and/or a pharmaceutically acceptable salt thereof is administered every 12 hours (q12 h).

9. The method according to claim 1, wherein 100 mg, 300 mg, or 500 mg of Compound I, a deuterated derivative thereof, and/or a pharmaceutically acceptable salt thereof is administered every 12 hours (q12 h).

10. The method according to claim 1, wherein the method comprises administering Compound I or a deuterated derivative thereof.

11. The method according to claim 1, wherein the method comprises administering a pharmaceutically acceptable salt of Compound I.

12. The method according to claim 1, wherein the Compound I, a deuterated derivative thereof, and/or a pharmaceutically acceptable salt thereof is comprised in a pharmaceutical composition.

13. The method according to claim 12, wherein the pharmaceutical composition is a tablet.

14. The method according to claim 13, wherein the tablet is suitable for oral administration.

15. The method according to claim 14, wherein the tablet for oral administration comprises 100 mg or 250 mg of Compound I, a deuterated derivative thereof, and/or a pharmaceutically acceptable salt thereof.

16. The method according to claim 15, wherein the tablet for oral administration comprises 100 mg of Compound I, a deuterated derivative thereof, and/or a pharmaceutically acceptable salt thereof.

17. The method according to claim 15, wherein the tablet for oral administration comprises 250 mg of Compound I, a deuterated derivative thereof, and/or a pharmaceutically acceptable salt thereof.

18. The method according to claim 13, wherein the tablet further comprises cellulose, croscarmellose sodium, and/or sodium stearyl fumarate.

19. The method according to claim 18, wherein the tablet comprises a coating comprising polyvinyl alcohol (PVA), polyethylene glycol (PEG), titanium dioxide, and talc.

20. The method according to claim 1, wherein the patient is in the fasted state.

21. The method according to claim 1, wherein the patient is in the fed state.
